(19) United States
(12) Patent Application Publication
(10) Pub. No.: US 2020/0254091 A1
(43) Pub. Date: Aug. 13, 2020

(54) Combination of a PARP Inhibitor and a PD-1 Axis Binding Antagonist

(71) Applicants: MERCK PATENT GMBH, Darmstadt (DE); Pfizer Inc., New York, NY (US)

(72) Inventors: John Andrew Blake-Haskins, San Marcos, CA (US); Christoffel Hendrik Boshoff, New York, NY (US); Rossano Cesari, Bologna (IT); Dimitry Serge Antoine Nuyten, Dallas, TX (US); Ross Anthony Stewart, Cambridge (GB); Fabian Zohren, Newton, MA (US)

(21) Appl. No.: 16/754,485

(22) PCT Filed: Oct. 10, 2018
(86) PCT No.: PCT/US2018/055174

§ 371 Date: Apr. 8, 2020

Related U.S. Application Data
(60) Provisional application No. 62/572,024, filed on Oct. 13, 2017.
(60) Provisional application No. 62/697,587, filed on Jul. 13, 2018.

(51) Int. Cl. A61K 39/395; A61P 35/00; A61K 31/5025

(52) U.S. Cl. CPC A61K 39/3955; A61K 2039/505; A61K 31/5025; A61P 35/00

(57) ABSTRACT

This invention relates to a method of treating cancer by administering a PARP inhibitor in combination with a PD-1 axis binding antagonist to a patient in need thereof.

### FIELD

The present invention relates to combination therapies useful for the treatment of cancer. In particular, this invention relates to methods for treating cancer by administering a PARP inhibitor in combination with a PD-1 axis binding antagonist. Pharmaceutical uses of the combination of the present invention are also described.

### BACKGROUND

PD-L1 is overexpressed in many cancers and is often associated with poor prognosis (Okazaki T et al., Intern. Immun. 2007 19(7):813) (Thompson R H et al., Cancer Res 2006, 66(7):3381). Interestingly, the majority of tumor infiltrating T lymphocytes predominantly express PD-1, in contrast to T lymphocytes in normal tissues and peripheral blood. PD-1 on tumor-reactive T cells can contribute to impaired antitumor immune responses (Ahmadzadeh et al, Blood 2009 1 14(8): 1537). This may be due to exploitation of PD-L1 signaling mediated by PD-L1 expressing tumor cells interacting with PD-1 expressing T cells to result in attenuation of T cell activation and evasion of immune surveillance (Sharpe et al., Nat Rev 2002) (Keir M E et al., 2008 Annu. Rev. Immunol. 26:677). Therefore, inhibition of the PD-L1/PD-1 interaction may enhance CD8+ T cell-mediated killing of tumors.

The inhibition of PD-1 axis signaling through its direct ligands (e.g., PD-L1, PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1. Optimal therapeutic treatment could combine blockade of PD-1 receptor/ligand interaction with other anti-cancer agents. There remains a need for such an optimal therapy for treating, stabilizing, preventing, and/or delaying development of various cancers.

Avelumab is a human immunoglobulin (Ig)G1 monoclonal antibody (mAb) directed against programmed death-ligand 1 (PD-L1). Avelumab selectively binds to PD-L1 and competitively blocks its interaction with programmed death receptor 1 (PD-1), thereby interfering with this key immune checkpoint inhibition pathway. Avelumab is the International Nonproprietary Name (INN) for the anti-PD-L1 monoclonal antibody MSB0010718C and has been described by its full length heavy and light chain sequences in WO2013079174, where it is referred to as A09-246-2. The glycosylation and truncation of the C-terminal Lysine in its heavy chain is described in European patent application No. 15198233.7.

In March 2017, avelumab received accelerated approval by the United States (US) Food and Drug Administration (FDA) as the first treatment for metastatic Merkel cell carcinoma (MCC). In May 2017, avelumab received accelerated approval by the US FDA for the treatment of patients with locally advanced or metastatic urothelial cancer (UC) with disease progression during or following platinum-containing chemotherapy, or within 12 months of neoadjuvant or adjuvant platinum-containing chemotherapy. Avelumab is currently being investigated as single agent and in combination with other anti-cancer therapies in patients with locally advanced or metastatic solid tumors and various hematological malignancies.

Poly (ADP-ribose) polymerase (PARP) engages in the naturally occurring process of DNA repair in a cell. PARP inhibition has been shown to be an effective therapeutic strategy against tumors associated with germline mutation in double-strand DNA repair genes by inducing synthetic lethality (Sonnenblick, A., et al., Nat Rev Clin Oncol, 2015. 12(1), 27-4). One PARP inhibitor (PARPi), olaparib, was approved by the U.S. Food and Drug Administration (FDA) in 2014 for the treatment of germline BRCA-mutated (gBRCAm) advanced ovarian cancer. More recently, other PARP inhibitors niraparib and rucaparib were also approved by FDA for treatment of ovarian cancer Talazoparib is a potent, orally available PARP inhibitor, which is cytotoxic to human cancer cell lines harboring gene mutations that compromise deoxyribonucleic acid (DNA) repair, an effect referred to as synthetic lethality, and by trapping PARP protein on DNA thereby preventing DNA repair, replication, and transcription.

The compound, talazoparib, which is “(8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one” and “(8S,9R)-5-fluoro-8-(4-fluorophenyl)-9-(1-methyl-1H-1,2,4-triazol-5-yl)-2,7,8,9-tetrahydro-3H-pyrido[4,3,2-de]phthalazin-3-one” (also referred to as “PF-06944076”, “MDV3800”, and “BMN673”) is a PARP inhibitor, having the structure,

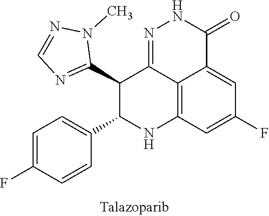 [SMILES: Cn1ncnc1[C@H]1c2n[nH]c(=O)c3cc(F)cc(c23)N[C@@H]1c1ccc(F)cc1]

Talazoparib, and pharmaceutically acceptable salts thereof, including the tosylate salt, are disclosed in International Publication Nos. WO 2010/017055 and WO 2012/054698. Additional methods of preparing talazoparib, and pharmaceutically acceptable salts thereof, including the tosylate salt, are described in International Publication Nos. WO 2011/097602, WO 2015/069851, and WO 2016/019125. Additional methods of treating cancer using talazoparib, and pharmaceutically acceptable salts thereof, including the tosylate salt, are disclosed in International Publication Nos. WO 2011/097334 and WO 2017/075091.

Talazoparib, as a single agent, has demonstrated efficacy, as well as an acceptable toxicity profile in patients with multiple types of solid tumors with DNA repair pathway abnormalities.

There remains a need of finding specific combination of specific PD-1 axis antagonist and specific PARP inhibitor to treat cancers, especially in specific patient populations, and potentially with optimized dosing regimens, to improve clinical anti-tumor activity as compared to the single agent treatment and to optimize the combination safety profile.

### SUMMARY

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein. Embodiments within an aspect as described below can be combined with any other embodiments not inconsistent within the same aspect.

In one embodiment, the invention is directed to a method for treating cancer comprising administering to a patient in need thereof an amount of a PARP inhibitor and an amount of a PD-1 axis binding antagonist, wherein the amounts together are effective in treating cancer. In one aspect of this embodiment and in combination with any other aspects not inconsistent, the PD-1 axis binding antagonist is a PD-L1 antibody, and in some embodiments, the PD-L1 antibody is avelumab. In another aspect of this embodiment and in combination with any other aspects not inconsistent, the PARP inhibitor is talazoparib, or a pharmaceutically acceptable salt thereof, and in one embodiment, the PARP inhibitor is talazoparib tosylate. In another aspect of this embodiment and in combination with any other aspects not inconsistent, the cancer is selected from the group consisting of non-small cell lung cancer (NSCLC), triple negative breast cancer (TNBC), hormone receptor positive breast cancer (HR+BC), ovarian cancer and preferably epithelial ovarian cancer, urothelial cancer (UC) and preferably transitional cell carcinoma of the urothelium of the bladder, urethra, ureters, or renal pelvis, and castration-resistant prostate cancer (CRPC). In another aspect of this embodiment and in combination with any other aspects not inconsistent, the amount of the PD-1 axis antagonist avelumab is intravenously about 10 mg/kg Q2W (one dose every two weeks), 10 mg/kg Q1W, 10 mg/kg Q1W for 12 weeks followed by 10 mg/kg Q2W, 800 mg Q2W, 1200 mg Q2W, or about 800 mg Q1W (one dose every week) for 12 weeks followed by about 800 mg Q2W, the amount of talazoparib, or a pharmaceutically acceptable salt thereof, is orally at a free base equivalent amount of about 0.5 mg, 0.75 mg or 1.0 mg QD (one dose daily), and in some embodiments, the amount of avelumab is about 800 mg Q2W. In one embodiment, the PARP inhibitor is talazoparib tosylate.

In another embodiment, the invention is directed to a method for treating cancer comprising administering to a patient in need thereof an amount of a PARP inhibitor talazoparib, or a pharmaceutically acceptable salt thereof, and an amount of a PD-1 axis binding antagonist avelumab, wherein the amounts together are effective in treating cancer. In an aspect of this embodiment, the PARP inhibitor is talazoparib tosylate. In one aspect of this embodiment and in combination with any other aspects not inconsistent, the cancer is DNA damage response (DDR) defect positive in at least one DDR genes selected from BRCA1, BRCA2, ATM, ATR and FANC, and in some embodiments, the cancer has a germline or somatic gene defect in BRCA1, BRCA2 or ATM. In one aspect of this embodiment and in combination with any other aspects not inconsistent, the cancer is determined to be DDR defect positive by the Foundation One® genetic profile assay. In another aspect of this embodiment and in combination with any other aspects not inconsistent, the cancer is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis to determine the LOH score include, without limitation, FoundationOne®, the Foundation Medicine® (Cambridge, Mass.) genomic profile assay and the Foundation Medicine T5 next generation sequencing assay. In another aspect of this embodiment and in combination with any other aspects not inconsistent, the patient has a homologous recombination deficiency (HRD) score of about 20 or above, 25 or above, 30 or above, 35 or above, 40 or above, 42 or above, 45 or above, or 50 or above. In some embodiments, the HRD score is determined by the Myriad Genetics myChoice® HRD or myChoice® HRD Plus assay. In another aspect of this embodiment and in combination with any other aspects not inconsistent, the patient has a tumor proportion score of less than about 1%, or equal or over about 1%, 5%, 10%, 25%, 50%, 75% or 80% for PD-L1. In another aspect of this embodiment and in combination with any other aspects not inconsistent, the amount of the PD-1 axis antagonist avelumab is intravenously about 10 mg/kg Q2W, 10 mg/kg Q1W, 10 mg/kg Q1W for 12 weeks followed by about 10 mg/kg Q2W, 800 mg Q2W, 1200 mg Q2W, or about 800 mg Q1W for 12 weeks followed by about 800 mg Q2W, the amount of talazoparib, or a pharmaceutically acceptable salt thereof, is administered orally at a free base equivalent amount of about 0.5 mg, 0.75 mg or 1.0 mg QD, and in some embodiments, the amount of avelumab is 800 mg Q2W. In one embodiment, the PARP inhibitor is talazoparib tosylate.

In another embodiment, the invention is directed to a method for treating cancer comprising administering to a patient in need thereof an amount of a PARP inhibitor talazoparib, or a pharmaceutically acceptable salt thereof, and in one embodiment, the PARP inhibitor is talazoparib tosylate, and an amount of a PD-1 axis binding antagonist avelumab, wherein the amounts together are effective in treating cancer and wherein the cancer is selected from the group consisting of non-small cell lung cancer, triple negative breast cancer, hormone receptor positive breast cancer, ovarian cancer and preferably epithelial ovarian cancer, urothelial cancer and preferably transitional cell carcinoma of the urothelium of the bladder, urethra, ureters, or renal pelvis, and castration-resistant prostate cancer. In one aspect of this embodiment and in combination with any other aspects not inconsistent, the cancer is DNA damage response (DDR) defect positive in at least one DDR genes selected from BRCA1, BRCA2, ATM, ATR and FANC, and in some embodiments, the cancer has a germline or somatic gene defect in BRCA1, BRCA2 or ATM. In one aspect of this embodiment and in combination with any other aspects not inconsistent, the cancer is determined to be DDR defect positive by genetic analysis using, for example without limitation, the FoundationOne genetic profile assay. In another aspect of this embodiment and in combination with any other aspects not inconsistent, the cancer is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes, for example without limitation, the Foundation Medicine genetic profile assay, and more the Foundation Medicine T5 next generation sequencing assay. In another aspect of this embodiment and in combination with any other aspects not inconsistent, the patient has a homologous recombination deficiency (HRD) score of about 20 or above, 25 or above, 30 or above, 35 or above, 40 or above, 42 or above, 45 or above, or 50 or above. In some aspects of this embodiment, the HRD score can be determined by the Myriad HRD or HRD Plus assay. In another aspect of this embodiment and in combination with any other aspects not inconsistent, the patient has a tumor proportion score of less than about 1%, or equal or over about 1%, 5%, 10%, 25%, 50%, 75% or 80% for PD-L1. In another aspect of this embodiment and in combination with any other aspects not inconsistent, the amount of the PD-1 axis antagonist avelumab is intravenously about 10 mg/kg Q2W, 10 mg/kg Q1W, 10 mg/kg Q1W for 12 weeks followed by about 10 mg/kg Q2W, 800 mg Q2W, 1200 mg Q2W, or about 800 mg Q1W for 12 weeks followed by about 800 mg Q2W, the amount of talazoparib, or a pharmaceutically acceptable salt thereof, is administered orally at a free base equivalent amount of about 0.5 mg, 0.75 mg or 1.0 mg QD, and preferably, the amount of avelumab is about 800 mg Q2W. In one embodiment, the PARP inhibitor is talazoparib tosylate.

In another embodiment, the invention is directed to a method of treating cancer, comprising administering to a patient in need thereof an amount of a PARP inhibitor and an amount of a PD-1 axis binding antagonist, wherein the PD-1 axis antagonist is avelumab, the PARP inhibitor is talazoparib or a pharmaceutically acceptable salt thereof, preferably a tosylate thereof, the amount of the PD-1 axis antagonist is intravenously about 10 mg/kg Q2W, 10 mg/kg Q1W, 10 mg/kg Q1W for 12 weeks followed by about 10 mg/kg Q2W, 800 mg Q2W, 1200 mg Q2W, or about 800 mg Q1W for 12 weeks followed by about 800 mg Q2W, the amount of talazoparib, or a pharmaceutically acceptable salt thereof, is administered orally at a free base equivalent amount of about 0.5 mg, 0.75 mg or 1.0 mg QD, and preferably, the amount of avelumab is about 800 mg Q2W. In one embodiment, the PARP inhibitor is talazoparib tosylate.

In one aspect of this embodiment and in combination with any other aspects not inconsistent, cancer is non-small cell lung cancer. In some embodiments of this aspect, the cancer is locally advanced or metastatic NSCLC, and the patient has received 0, 1 or 2 prior lines of platinum-based chemotherapy treatment for the locally advanced or metastatic NSCLC and had no progression while on such chemotherapy treatment for the locally advanced or metastatic NSCLC, and that the cancer has no EFGR, ALK or ROS-1 genomic tumor aberrations. Exemplary platinum-based chemotherapy includes, without limitation, platinum-based doublets and docetaxel. In some embodiments of this aspect, the cancer is DDR defect positive in at least one DDR gene selected from BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the ovarian cancer patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and more preferably the genetic analysis is Foundation Medicine T5 next generation sequencing assay. In some embodiments of this aspect, the ovarian cancer patient has a homologous recombination deficiency (HRD) score of about 20 or above, 25 or above, 30 or above, 35 or above, 40 or above, 42 or above, 45 or above, or 50 or above, and preferably, the HRD score is determined by the Myriad HRD or HRD Plus assay. In some embodiments of this aspect, the NSCLC patient has a tumor proportion score of less than about 1%, equal or over about 1%, 5%, 10%, 25%, 50%, 75% or 80% for PD-L1.

In another aspect of this embodiment and in combination with any other aspects not inconsistent, cancer is ovarian cancer. In some embodiments of this aspect, the cancer is epithelial ovarian cancer. In some embodiments of this aspect, the cancer is locally advanced or metastatic ovarian cancer, and the patient has had 1 or 2 prior lines of platinum-based chemotherapy with no disease progression during or within 6 months after receiving the last dose of the platinum-based chemotherapy (platinum sensitive). Exemplary platinum-based chemotherapy includes, without limitation, cisplatin or, carboplatin, both in combination with a taxane. In some embodiments of this aspect, the cancer is DDR defect positive in at least one DDR gene selected from BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the ovarian cancer patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and more preferably the genetic analysis is Foundation Medicine T5 next generation sequencing assay. In some embodiments of this aspect, the ovarian cancer patient has a homologous recombination deficiency (HRD) score of about 20 or above, 25 or above, 30 or above, 35 or above, 40 or above, 42 or above, 45 or above, or 50 or above, and preferably, the HRD score is determined by the Myriad HRD or HRD Plus assay. In some embodiments of this aspect, the ovarian cancer patient has a tumor proportion score of less than about 1%, equal or over about 1%, 5%, 10%, 25%, 50%, 75% or 80% for PD-L1.

In another aspect of this embodiment and in combination with any other aspects not inconsistent, cancer is urothelial cancer. In some embodiments of this aspect, the cancer is locally advanced or metastatic urothelial cancer, wherein the patient has received 0, 1 or 2 prior systemic lines of platinum-based chemotherapy with no progression while on the prior treatment with platinum-based chemotherapy. Examplary platinum-based chemotherapy includes, without limitation, gemcitabine in combination with cisplatin, or carboplatin. In some embodiments of this aspect, the cancer is DDR defect positive in at least one DDR gene selected from the group consisting of BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and the Foundation Medicine T5 next generation sequencing assay. In some embodiments of this aspect, the patient has a homologous recombination deficiency (HRD) score of about 20 or above, 25 or above, 30 or above, 35 or above, 40 or above, 42 or above, 45 or above, or 50 or above, and in some embodiments, the HRD score can be determined by, for example without limitation, the Myriad HRD or HRD Plus assay. In some embodiments of this aspect the patient has a tumor proportion score of less than about 1%, equal or over about 1%, 5%, 10%, 25%, 50%, 75% or 80% for PD-L1.

In another aspect of this embodiment and in combination with any other aspects not inconsistent, cancer is castration-resistant prostate cancer (CRPC). In some embodiments of this aspect, the cancer is locally advanced or metastatic CRPC, the patient has received 1 or 2 prior chemotherapy treatments including at least 1 taxane-based chemotherapy treatment, after progressed on at least 1 line of prior novel hormonal therapy treatment. Exemplary taxane-based chemotherapy treatment includes, without limitation, docetaxel or cabazitaxel. Exemplary hormonal therapy treatment includes, without limitation, the combination of enzalutamide and prednisone, and the combination of abiraterone acetate and prednisone. In some embodiments of this aspect, the cancer is DDR defect positive in at least one DDR gene selected from the group consisting of BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the CRPC patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and the Foundation Medicine T5 next generation sequencing assay. In some embodiments of this aspect, the CRPC patient has a homologous recombination deficiency (HRD) score of about 20 or above, 25 or above, 30 or above, 35 or above, 40 or above, 42 or above, 45 or above, or 50 or above, and in some embodiments, the HRD score can be determined by, for example without limitation, the Myriad HRD or HRD Plus assay. In some embodiments of this aspect, the CRPC patient has a tumor proportion score of less than about 1%, equal or over about 1%, 5%, 10%, 25%, 50%, 75% or 80% for PD-L1.

In another aspect of this embodiment and in combination with any other aspects not inconsistent, the cancer is breast cancer. In some embodiments, the cancer is triple negative breast cancer (TNBC) or hormone receptor positive (HR+) breast cancer. In some embodiments of this aspect, the cancer is locally advanced or metastatic TNBC, wherein the TNBC patient has had 0, 1 or 2 prior lines of chemotherapy treatment for the locally advanced or metastatic TNBC, with no progression while on the prior chemotherapy treatment when the prior chemotherapy treatment is platinum-based chemotherapy, or with no progression while on the prior chemotherapy treatment or within 6 months after stopping the prior chemotherapy treatment, when the prior chemotherapy treatment is adjuvant or neoadjuvant platinum-based chemotherapy. Exemplary prior chemotherapy treatments include, but are not limited to anthracyclines, taxanes, gemcitabine, capecitabine, vinorelbine and platinum-based chemotherapy. In some embodiments of this aspect, the cancer is locally advanced or metastatic HR+ breast cancer, wherein the patient has received 0, 1 or 2 prior lines of chemotherapy treatment for the locally advanced or metasatic HR+ breast cancer following progression from standard hormone therapy. Exemplary chemotherapy treatments include, without limitation, anthracyclines, taxanes, gemcitabine, capecitabine, vinorelbine, and platinum-based chemotherapy. In some embodiments of this aspect, the cancer is DDR defect positive in at least one DDR gene selected from the group consisting of BRCA1, BRCA2, ATM, ATR and FANC. In some embodiments of this aspect, the cancer is DDR defect positive in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by, for example, without limitation the FoundationOne assay. In some embodiments of this aspect, the TNBC or HR+BR patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay and the Foundation Medicine T5 next generation sequencing assay. In some embodiments of this aspect, the TNBC or HR+BR patient has a homologous recombination deficiency (HRD) score of about 20 or above, 25 or above, 30 or above, 35 or above, 40 or above, 42 or above, 45 or above, or 50 or above, and in some embodiments, the HRD score can be determined by, for example without limitation, the Myriad HRD or HRD Plus assay. In some embodiments of this aspect, the cancer has a tumor proportion score of less than about 1%, equal or over about 1%, 5%, 10%, 25%, 50%, 75% or 80% for PD-L1.

In another embodiment, the invention is directed to all of the methods of treating cancer as described above, wherein the PD-1 axis binding antagonist is avelumab and the PARP inhibitor is talazoparib, or a pharmaceutically acceptable salt thereof, preferably a tosylate thereof, the methods further comprising: administering to the patient an amount of a chemotherapeutic agent or radiotherapy, wherein the amounts together are effective in treating cancer.

In another embodiment, the invention is directed to a method for treating cancer comprising a first treatment regimen followed by a second treatment regimen, wherein the first treatment regimen comprises administering to a patient in need thereof an amount of a chemotherapy and an amount of a PD-1 axis binding antagonist, wherein the second treatment regimen comprises administering to the patient in need thereof an amount of a PARP inhibitor and an amount of a PD-1 axis binding antagonist. In one aspect of this embodiment, the amounts together are effective in treating cancer. For clarify, the “amounts together” herein refers to the amount of chemotherapy, the amount of the PD-1 axis binding antagonist in the first treatment regimen, the amount of the PARP inhibitor and the amount of the PD-1 axis binding antagonist in the second treatment regimen, all together. The other aspects of this embodiment, and the embodiments within each aspect, will be the same as the ones of the embodiment (of the invention) immediately following this paragraph.

In another embodiment, the invention is directed to a method for treating cancer comprising a first treatment regimen followed by a second treatment regimen, wherein the first treatment regimen comprises administering to a patient in need thereof an amount of a chemotherapy and an amount of a PD-1 axis binding antagonist, wherein the second treatment regimen comprises administering to the patient in need thereof an amount of a PARP inhibitor and an amount of a PD-1 axis binding antagonist, wherein the amounts together are effective in treating cancer. For clarify, “the amounts together” herein refers to the amount of chemotherapy, the amount of the PD-1 axis binding antagonist in the first treatment regimen, the amount of the PARP inhibitor and the amount of the PD-1 axis binding antagonist in the second treatment regimen, all together.

In one aspect of this embodiment and in combination with any other aspects not inconsistent, the first treatment regimen comprises administering to the patient in need thereof the amount of the chemotherapy and the amount of the PD-1 axis binding antagonist for at least one cycle of a first treatment cycle. In some embodiments of this aspect, the first treatment cycle is a two week cycle or a three week cycle and the patient is treated for at least 2 cycles, at least 3 cycles, at least 4 cycles, at least 5 cycles or at least 6 cycles of the first treatment cycle. In some embodiments of this aspect, the first treatment cycle is a three week cycle and the patient is treated for at least 6 cycles of the first treatment cycle.

In another aspect of this embodiment and in combination with any other aspects not inconsistent, the the second treatment regimen comprises administering to the patient in need thereof the amount of the PARP inhibitor and the amount of the PD-1 axis binding antagonist for at least one cycle of a second treatment cycle. In some embodiments of this aspect, the second treatment cycle is a three week cycle, a four week cycle, a five week cycle or a six week cycle, and the patient is treated for at least 1 cycle, at least 2 cycles, at least 3 cycles or at least 4 cycles of the second treatment cycle. In some embodiments of this aspect, the second treatment cycle is a six weeks cycle and the patient is treated for at least one cycle of the second treatment cycle.

In another aspect of this embodiment and in combination with any other aspects not inconsistent, the cancer is ovarian cancer, and in particular, locally advanced or metastatic ovarian cancer. In another aspect of this embodiment and in combination with any other aspects not inconsistent, the cancer is epithelial ovarian, fallopian tube or primary peritoneal cancer, and in particular, stage III-IV epithelial ovarian, fallopian tube or primary peritoneal cancer. In another aspect of this embodiment and in combination with any other aspects not inconsistent, the cancer is stage III-IV epithelial ovarian cancer. In another aspect of this embodiment and in combination with any other aspects not inconsistent, the patient has not received any prior systemic anti-cancer therapy or radiotherapy with respect to the cancer. Exemplary systemic anti-cancer therapy includes, but is not limited to chemotherapy, anti-VEGF antibodies, PARP inhibitors, interleukin-2, interferon alpha, PD-L1 axis binding antagonist, anti-CD137 antibody, anti-cytotoxic T-lymphocyte associated antigen 4 (anti CTLA4) antibody, VEGF inhibitors, cancer vaccines and oncolytic vaccines.

In another aspect of this embodiment and in combination with any other aspects not inconsistent, the chemotherapy is a platinum-based chemotherapy, the PD-1 axis binding antagonist in the first treatment regimen is avelumab; the PD-1 axis binding antagonist in the second treatment regimen is avelumab, and the PARP inhibitor in the second treatment regimen is talazoparib, or a pharmaceutically acceptable salt thereof. In some embodiments of this aspect, the PARP inhibitor is talazoparib tosylate. In some embodiments of this aspect, the platinum-based chemotherapy is a platinum-based doublet. In some embodiments of this aspect, the platinum-based doublet is paclitaxel and carboplatin.

In another aspect of this embodiment and in combination with any other aspects not inconsistent, the chemotherapy is paclitaxel and carboplatin, the PD-1 axis binding antagonist in the first treatment regimen is avelumab; the PD-1 axis binding antagonist in the second treatment regimen is avelumab, and the PARP inhibitor in the second treatment regimen is talazoparib tosylate.

In another aspect of this embodiment and in combination with any other aspects not inconsistent, the first treatment cycle is a three week cycle, the chemotherapy is paclitaxel and carboplatin, paclitaxel is administered intravenously in the amount of about 110 mg/m² to about 175 mg/m², on day 1 of the first treatment cycle for 6 cycles, carboplatin is administered intravenously in the amount of about calculated AUC 3 dose to about calculated AUC 6 dose, on day 1 of the first treatment cycle for 6 cycles; the PD-1 axis binding antagonist in the first treatment regimen is avelumab, and is administered intravenously in the amount of about 700 mg, 750 mg, 800 mg, 850 mg or 900 mg on day 1 of the first treatment cycle for 6 cycles, and the PARP inhibitor and the PD-1 axis binding antagonist of the second treatment regimen are administered in a second treatment cycle.

In some embodiments of this aspect, paclitaxel is administered intravenously in the amount of about 175 mg/m², 135 mg/m² or 110 mg/m² on day 1 of the first treatment cycle for 6 cycles. In some embodiments of this aspect, carboplatin is administered intravenously in the amount of about a calculated AUC 3 dose, a calculated AUC 4 dose, a calculated AUC 5 dose or a calculated AUC 6 dose, on day 1 of the first treatment cycle for 6 cycles.

In some embodiments of this aspect, the second treatment cycle is a six week cycle, the PARP inhibitor is talazoparib tosylate, and is administered in the amount of about 0.25 mg, 0.5 mg, 0.75 mg or 1.0 mg orally once per day, the PD-1 axis binding antagonist of the second treatment regimen is avelumab and is administered intravenously in the amount of about 700 mg, 750 mg, 800 mg, 850 mg, or 900 mg on day 1, day 15 and day 29 of each of the second treatment cycle.

In some embodiments of this aspect, paclitaxel is administered in the amount of about 175 mg/m² on day 1 of the first treatment cycle for 6 cycles, carboplatin is administered in the amount of about calculated AUC 6 dose or calculated AUC 5 dose on day 1 of the first treatment cycle for 6 cycles; the PD-1 axis binding antagonist in the first treatment regimen is avelumab, and is administered intravenously in the amount of about 800 mg on day 1 of the first treatment cycle for 6 cycles.

In some embodiments of this aspect, the PARP inhibitor is talazoparib tosylate, and is administered in the amount of about 1.0 mg orally once per day, the PD-1 axis binding antagonist of the second treatment regimen is avelumab and is administered intravenously in the amount of about 800 mg on day 1, day 15 and day 29 of each of the second treatment cycle.

In some embodiments of this aspect, the cancer is locally advanced or metastatic ovarian cancer. In some embodiments of this aspect, the cancer is stage III-IV epithelial ovarian, fallopian tube or primary peritoneal cancer. In some embodiments of this aspect, the cancer is stage III-IV ovarian cancer. In some embodiments of this aspect, the patient has not received any prior treatment of systemic anti-cancer therapy or radiotherapy with respect to the cancer. Exemplary systemic anti-cancer therapy includes, but is not limited to interleukin-2, interferon alpha, PD-L1 axis binding antagonist, anti-CD137 antibody, anti-cytotoxic T-lymphocyte associated antigen 4 (anti CTLA4) antibody, VEGF inhibitors, cancer vaccines and oncolytic vaccines. In some embodiments of this aspect, the cancer is DDR defect positive. In some embodiments of this aspect, the cancer is DDR defect positive in at least one DDR gene selected from the group consisting of BRCA1 and BRCA2.

In one aspect of this embodiment and in combination with any other aspects not inconsistent, the cancer is DDR defect positive in at least one DDR genes selected from BRCA1, BRCA2, ATM, ATR and FANC, and in some embodiments, the cancer has a germline or somatic gene defect in BRCA1, BRCA2 In one aspect of this embodiment and in combination with any other aspects not inconsistent, the cancer is determined to be DDR defect positive by genetic analysis using, for example without limitation, the FoundationOne genetic profile assay. In another aspect of this embodiment and in combination with any other aspects not inconsistent, the cancer is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Even more preferred LOH score indicative of deficiency in DNA damage repair includes about 16% or more. Exemplary genetic analysis includes, for example without limitation, the Foundation Medicine genetic profile assay, and preferably the Foundation Medicine T5 next generation sequencing assay, and more preferably the Foundation Focus CDx BRCA LOH test. In another aspect of this embodiment and in combination with any other aspects not inconsistent, the patient has a homologous recombination deficiency (HRD) score of about 20 or above, 25 or above, 30 or above, 35 or above, 40 or above, 42 or above, 45 or above, or 50 or above. In some aspects of this embodiment, the HRD score can be determined by the Myriad HRD or HRD Plus assay. In another aspect of this embodiment and in combination with any other aspects not inconsistent, the patient has a tumor proportion score of less than about 1%, or equal or over about 1%, 5%, 10%, 25%, 50%, 75% or 80% for PD-L1.

In another embodiment, the invention is directed to any of the methods of treating cancer as described above, wherein the PD-1 axis binding antagonist is avelumab and the PARP inhibitor is talazoparib, or a pharmaceutically acceptable salt thereof, preferably a tosylate thereof, wherein the treatment provides a therapeutic effect as indicated by a tumor response evaluation criteria including but not limited to objective response rate, complete response rate, progression free survival, duration of response, r duration of stable disease, immune-related objective response rate, immune-related complete response rate, immune-related progression free survival, immune-related duration of response, or immune-related duration of stable disease. In one aspect of this embodiment and in combination with any other aspects not inconsistent, the therapeutic effect is indicated by objective response rate or immune-related objective response rate of equal or higher than about 20%, 25%, 30%, 35%, 40%, 45%, 50%, 55% or 60%, 65%, 70%, 75% or 80%. In another aspect of this embodiment and in combination with any other aspects not inconsistent, the therapeutic effect is indicated by progression free survival or immune-related progression free survival of equal or more than about 4 months, 5 months, 6 months, 7 months, 8 months, 9 months, 10 months, 11 months 12 months, 13 months, 14 months, 15 months, 16 months, 17 months, 18 months, 19 months, 20 months, or 2 years. In another aspect of this embodiment and in combination with any other aspects not inconsistent, the therapeutic effect is indicated by duration of response immune-related duration of response of equal or more than about 4 months, 5 months, 6 months, 7 months, 8 months, 9 months, 10 months 11 months, 12 months, 13 months, 14 months, 15 months, 16 months, 17 months, 18 months, 19 months, 20 months, or 2 years. In another In another aspect of this embodiment and in combination with any other aspects not inconsistent, the treatment further provides an advantage as indicated by less occurrence of drug related toxicity or immune-related adverse events, or less occurrence of drug related toxicity or immune-related adverse events of equal or above grade 3, as compared to other similar treatments in the art.

In another embodiment, the invention is directed to a method for treating cancer comprising administering to a patient in need thereof an amount of a PARP inhibitor talazoparib, or a pharmaceutically acceptable salt thereof, preferably a tosylate thereof, and an amount of a PD-1 axis binding antagonist RN888, wherein the amounts together are effective in treating cancer. In one aspect of this embodiment and in combination with any other aspects not inconsistent, the cancer is DNA damage response defect positive in at least one DDR genes selected from BRCA1, BRCA2, ATM, ATR and FANC, and preferably, the cancer has a germline or somatic gene defect in BRCA1, BRCA2 or ATM. In one aspect of this embodiment and in combination with any other aspects not inconsistent, the cancer is determined to be DDR defect positive by genetic analysis using, for example without limitation, the FoundationOne genetic profile assay. In another aspect of this embodiment and in combination with any other aspects not inconsistent, the cancer is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes without limitation the Foundation Medicine genetic profile assay, and the Foundation Medicine T5 next generation sequencing assay. In another aspect of this embodiment and in combination with any other aspects not inconsistent, the patient has a homologous recombination deficiency (HRD) score of about 20 or above, 25 or above, 30 or above, 35 or above, 40 or above, 42 or above, 45 or above, or 50 or above, and preferably, the HRD score is determined by, for example without limitation, the Myriad HRD or HRD Plus assay. In another aspect of this embodiment and in combination with any other aspects not inconsistent, the patient has a tumor proportion score of less than about 1%, equal or over about 1%, 5%, 10%, 25%, 50%, 75% or 80% for PD-L1. In another aspect of this embodiment and in combination with any other aspects not inconsistent, the amount of the PD-1 axis axis antagonist RN888 is subcutaneously about 300 mg Q4W (one dose every four weeks), and the amount of talazoparib, or a pharmaceutically acceptable salt thereof, is administered orally at a free base equivalent amount of about 0.5 mg, 0.75 mg or 1.0 mg QD. In one embodiment, the PARP inhibitor is talazoparib tosylate.

In another embodiment, the invention is directed to the method of treating cancer comprising administering to a patient an amount of a PD-1 axis binding antagonist RN888, and an amount of a PARP inhibitor talazoparib, or a pharmaceutically acceptable salt thereof, and preferably a tosylate thereof, wherein the amounts together are effective in treating cancer. In one aspect of this embodiment and in combination with any other aspects not inconsistent, the cancer is selected from the group consisting of non-small cell lung cancer, triple negative breast cancer, hormone receptor positive breast cancer, ovarian cancer, urothelial cancer and castration-resistant prostate cancer. In another aspect of this embodiment and in combination with any other aspects not inconsistent, the cancer is DNA damage response (DDR) defect positive in at least one DDR genes selected from BRCA1, BRCA2, ATM, ATR and FANC. In some embodiments, the cancer has a germline or somatic gene defect in BRCA1, BRCA2 or ATM. In some embodiments, the cancer is determined to be DDR defect positive can be determined by, for example without limitation, the FoundationOne generic profile assay. In another aspect of this embodiment and in combination with any other aspects not inconsistent, the patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes without limitation the Foundation Medicine genetic profile assay, and the Foundation Medicine T5 next generation sequencing assay. In another aspect of this embodiment and in combination with any other aspects not inconsistent, the patient has a homologous recombination deficiency (HRD) score of about 20 or above, 25 or above, 30 or above, 35 or above, 40 or above, 42 or above, 45 or above, or 50 or above, and preferably, the HRD score can be determined by for example without limitation the Myriad HRD or HRD Plus assay. In another aspect of this embodiment and in combination with any other aspects not inconsistent, the patient has a tumor proportion score of less than about 1%, equal or over about 1%, 5%, 10%, 25%, 50%, 75% or 80% for PD-L1. In another aspect of this embodiment and in combination with any other aspects not inconsistent, the amount of the PD-1 axis antagonist RN888 is subcutaneously about 300 mg Q4W, and the amount of talazoparib, or a pharmaceutically acceptable salt thereof, is administered orally at a free base equivalent amount of about 0.5 mg, 0.75 mg or 1.0 mg QD. In one embodiment, the PARP inhibitor is talazoparib tosylate.

In another embodiment, the invention is directed to a method of treating cancer, comprising administering to a patient in need thereof an amount of a PARP inhibitor and an amount of a PD-1 axis binding antagonist, wherein the PD-1 axis antagonist is RN888, the PARP inhibitor is talazoparib or a pharmaceutically acceptable salt thereof, and preferably a tosylate thereof, the amount of the PD-1 axis antagonist RN888 subcutaneously about 250 mg, 300 mg, 350 mg, or 400 mg, each of which Q3W or Q4W, but preferably about 300 mg Q4W, the amount of talazoparib or a pharmaceutically acceptable salt thereof, is administered orally at a free base equivalent amount of about 0.5 mg, 0.75 mg or 1.0 mg QD. In one embodiment, the PARP inhibitor is talazoparib tosylate.

In one aspect of this embodiment and in combination with any other aspects not inconsistent, the cancer is non-small cell lung cancer. In some embodiments of this aspect, the cancer is locally advanced or metastatic NSCLC, and the patient has received 0, 1 or 2 prior lines of platinum-based chemotherapy treatment for the locally advanced or metastatic NSCLC and had no progression while on such chemotherapy treatment for the locally advanced or metastatic NSCLC, and that the cancer has no EFGR, ALK or ROS-1 genomic tumor aberrations. Exemplary platinum-based chemotherapy includes, without limitation, platinum-based doublets and docetaxel. In some embodiments of this aspect, the cancer is DDR defect positive in at least one DDR gene selected from BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the ovarian cancer patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and more preferably the genetic analysis is Foundation Medicine T5 next generation sequencing assay. In some embodiments of this aspect, the ovarian cancer patient has a homologous recombination deficiency (HRD) score of about 20 or above, 25 or above, 30 or above, 35 or above, 40 or above, 42 or above, 45 or above, or 50 or above, and preferably, the HRD score is determined by the Myriad HRD or HRD Plus assay. In some embodiments of this aspect, the NSCLC patient has a tumor proportion score of less than about 1%, equal or over about 1%, 5%, 10%, 25%, 50%, 75% or 80% for PD-L1.

In another aspect of this embodiment and in combination with any other aspects not inconsistent, the cancer is ovarian cancer. In some embodiments of this aspect, the cancer is locally advanced or metastatic ovarian cancer, and the patient has had 1 or 2 prior lines of platinum-based chemotherapy with no disease progression during or within 6 months after receiving the last dose of the platinum-based chemotherapy (platinum sensitive). Exemplary platinum-based chemotherapy includes, without limitation, cisplatin or carboplatin, both in combination with a taxane. In some embodiments of this aspect, the cancer is DDR defect positive in at least one DDR gene selected from BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by, for example without limitation, the FoundationOne assay. In some embodiment of this aspect, the patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes without limitation the Foundation Medicine genetic profile assay and the Foundation Medicine T5 next generation sequencing assay. In some embodiments of this aspect, the patient has a homologous recombination deficiency (HRD) score of about 20 or above, 25 or above, 30 or above, 35 or above, 40 or above, 42 or above, 45 or above, or 50 or above, and preferably, the HRD score can be determined by for example without limitation the Myriad HRD or HRD Plus assay.

In another aspect of this embodiment and in combination with any other aspects not inconsistent, cancer is urothelial cancer. In some embodiments of this aspect, the cancer is advanced or metastatic urothelial cancer, wherein the patient has received 0-0, 1, or 2 prior systemic lines of platinum-based chemotherapy with no progression while on the prior treatment with platinum-based chemotherapy. Examplary platinum-based chemotherapy includes, without limitation, gemcitabine in combination with cisplatin, or carboplatin. In some embodiments of this aspect, the cancer is DDR defect positive in at least one DDR gene selected from the group consisting of BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and the Foundation Medicine T5 next generation sequencing assay. In some embodiments of this aspect, the patient has a homologous recombination deficiency (HRD) score of about 20 or above, 25 or above, 30 or above, 35 or above, 40 or above, 42 or above, 45 or above, or 50 or above, and in some embodiments, the HRD score can be determined by, for example without limitation, the Myriad HRD or HRD Plus assay. In some embodiments of this aspect the patient has a tumor proportion score of less than about 1%, equal or over about 1%, 5%, 10%, 25%, 50%, 75% or 80% for PD-L1.

In another aspect of this embodiment and in combination with any other aspects not inconsistent, cancer is castration-resistant prostate cancer (CRPC). In some embodiments of this aspect, the patient has received 0, 1 or 2 prior chemotherapy treatments including at least 1 taxane-based chemotherapy treatment, after progressed on at least 1 line of novel hormonal therapy treatment. Exemplary taxane-based chemotherapy treatment includes, without limitation, docetaxel or cabazitaxel. Exemplary hormonal therapy treatment includes, without limitation, the combination of enzalutamide and prednisone, and the combination of abiraterone acetate and prednisone. In some embodiments of this aspect, the cancer is DDR defect positive in at least one DDR gene selected from the group consisting of BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the cancer is DDR defect positive in at least one DDR gene selected from the group consisting of BRCA1 or BRCA2. In some embodiments of this aspect, the cancer is determined to be DDR defect positive by genetic analysis using, for example without limitation, the FoundationOne assay. In some embodiments of this aspect, the CRPC patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes for example without limitation the Foundation Medicine genetic profile assay, and the Foundation Medicine T5 next generation sequencing assay. In some embodiments of this aspect, the CRPC patient has a homologous recombination deficiency (HRD) score of about 20 or above, 25 or above, 30 or above, 35 or above, 40 or above, 42 or above, 45 or above, or 50 or above, and in some embodiments, the HRD score can be determined by, for example without limitation, the Myriad HRD or HRD Plus assay.

In another aspect of this embodiment and in combination with any other aspects not inconsistent, cancer is breast cancer. In some embodiments of this aspect, the breast cancer is triple negative breast cancer or hormone receptor positive breast cancer. In some embodiments of this aspect, the cancer is locally advanced or metastatic TNBC, wherein the TNBC patient has had 0, 1 or 2 prior lines of chemotherapy treatment for the locally advanced or metastatic TNBC, with no progression while on the prior chemotherapy treatment when the prior chemotherapy treatment is platinum-based chemotherapy, or with no progression while on the prior chemotherapy treatment or within 6 months after stopping the prior chemotherapy treatment, when the prior chemotherapy treatment is adjuvant or neoadjuvant platinum-based chemotherapy. Exemplary prior chemotherapy treatments include, but are not limited to anthracyclines, taxanes, gemcitabine, capecitabine, vinorelbine and platinum-based chemotherapy. In some embodiments of this aspect, the cancer is locally advanced or metastatic HR+ breast cancer, wherein the patient has received 0, 1 or 2 prior lines of chemotherapy treatment for the locally advanced or metasatic HR+ breast cancer following progression from standard hormone therapy. Exemplary chemotherapy treatments include, without limitation, anthracyclines, taxanes, gemcitabine, capecitabine, vinorelbine, and platinum-based chemotherapy. In some embodiments of this aspect, the cancer is DDR defect positive in at least one DDR gene selected from the group consisting of BRCA1, BRCA2, ATM, ATR and FANC. In some embodiments of this aspect, the cancer is DDR defect positive in in at least one DDR gene selected from the group consisting of BRCA1, BRCA2, and ATM. In some embodiments of this aspect, the HR+ or TNBC patient is DDR defect positive in at least one DDR gene selected from the group consisting of BRCA1 or BRCA2. In some embodiments of this aspect, the cancer is determined to be DDR defect positive by, for example without limitation, the FoundationOne assay. In some embodiments of this aspect, the patient is determined to have Loss of heterozygosity (LOH) score indicative of deficiency in DNA damage repair by a genetic analysis. Preferred LOH score indicative of deficiency in DNA damage repair includes about 5% or more, 10% or more, 15% or more, 20% or more, and 25% or more. More preferred LOH score indicative of deficiency in DNA damage repair includes about 14% or more. Exemplary genetic analysis includes without limitation the Foundation Medicine genetic profile assay and the Foundation Medicine T5 next generation sequencing assay. In another aspect of this embodiment and in combination with any other aspects not inconsistent, the patient has a homologous recombination deficiency (HRD) score of about 20 or above, 25 or above, 30 or above, 35 or above, 40 or above, 42 or above, 45 or above, or 50 or above, and preferably, the HRD score can be determined by for example without limitation the Myriad HRD or HRD Plus assay.

In another aspect of this embodiment and in combination with one of the any other aspects of this embodiment, the patient has a tumor proportion score of less than about 1%, equal or over about 1%, 5%, 10%, 25%, 50%, 75% or 80% for PD-L1.

In another embodiment, the invention is directed to all of the methods of treating cancer as described in the preceding paragraphs under the subtitle of “SUMMARY”, wherein the PD-1 axis binding antagonist is RN888 and the PARP inhibitor is talazoparib, or a pharmaceutically acceptable salt thereof, and preferably a tosylate thereof, and the method further comprising administering to the patient an amount of a chemotherapeutic agent or radiotherapy, wherein the amounts together are effective in treating cancer.

In another embodiment, the invention is directed to any of the methods of treating cancer as described above, wherein the PD-1 axis binding antagonist is RN888 and the PARP inhibitor is talazoparib, or a pharmaceutically acceptable salt thereof, preferably a tosylate thereof, wherein the treatment provides a therapeutic effect as indicated by a tumor response evaluation criteria including but not limited to objective response rate, complete response rate, progression free survival, duration of response, r duration of stable disease, immune-related objective response rate, immune-related complete response rate, immune-related progression free survival, immune-related duration of response, or immune-related duration of stable disease. In one aspect of this embodiment and in combination with any other aspects not inconsistent, the therapeutic effect is indicated by objective response rate or immune-related objective response rate of equal or higher than about 20%, 25%, 30%, 35%, 40%, 45%, 50%, 55% or 60%. In another aspect of this embodiment and in combination with any other aspects not inconsistent, the therapeutic effect is indicated by progression free survival or immune-related progression free survival of equal or more than about 4 months, 5 months, 6 months, 7 months, 8 months, 9 months, 10 months, 11 months 12 months, 13 months, 14 months, 15 months, 16 months, 17 months, 18 months, 19 months, 20 months, or 2 years. In another aspect of this embodiment and in combination with any other aspects not inconsistent, the therapeutic effect is indicated by duration of response immune-related duration of response of equal or more than about 4 months, 5 months, 6 months, 7 months, 8 months, 9 months, 10 months 11 months, 12 months, 13 months, 14 months, 15 months, 16 months, 17 months, 18 months, 19 months, 20 months, or 2 years. In another In another aspect of this embodiment and in combination with any other aspects not inconsistent, the treatment further provides an advantage as indicated by less occurrence of drug related toxicity or immune-related adverse events, or less occurrence of drug related toxicity or immune-related adverse events of equal or above grade 3, as compared to other similar treatments in the art.

### DETAILED DESCRIPTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

### General Techniques and Definitions

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., 1RL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a PARP inhibitor or PD-1 axis binding antagonist, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, and rabbit) and most preferably a human. “Treatment”, as used in a clinical setting, is intended for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolong survival of patients having a disease (e.g., cancer).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-L1). Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., PD-L1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-L1 epitope is an antibody that binds this epitope with greater affinity, avidity, more readily, and/or with greater duration than it binds to other PD-L1 epitopes or non-PD-L1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Iazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original | |
|---|---|
| residue | Conservative substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “PD-1 axis binding antagonist” as used herein refers to a molecule that inhibits the interaction of a PD-1 axis binding partner with either one or more of its binding partner, so as to remove T-cell dysfunction resulting from signaling on the PD-1 signaling axis, with a result being to restore or enhance T-cell function. As used herein, a PD-1 axis binding antagonist includes a PD-1 binding antagonist, a PD-L1 binding antagonist and a PD-L2 binding antagonist.

Table 2 below provides a list of the amino acid sequences of exemplary PD-1 axis binding antagonists for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE 2

| | |
|---|---|
| mAb7(aka RN888) | QVQLVQSGAEVKKPGASVKVSCKASGYTFTSYWINWVRQAPGQGL |
| or mAb15 full- | EWMGNIYPGSSLTNYNEKFKNRVTMTRDTSTSTVYMELSSLRSEDT |
| length heavy chain | AVYYCARLSTGTFAYWGQGTLVTVSSASTKGPSVFPLAPCSRSTSE |
| | STAALGCLVKDYFPEPVTVSWNSGALTSGVHTFPAVLQSSGLYSLSS |
| | VVTVPSSSLGTKTYTCNVDHKPSNTKVDKRVESKYGPPCPPCPAPE |
| | FLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSN |
| | KGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQ |
| | EGNVFSCSVMHEALHNHYTQKSLSLSLGK |
| | (SEQ ID NO: 1) |
| mAb7 or mAb 15 | QVQLVQSGAEVKKPGASVKVSCKASGYTFTSYWINWVRQAPGQGL |
| full-length heavy | EWMGNIYPGSSLTNYNEKFKNRVTMTRDTSTSTVYMELSSLRSEDT |
| chain without the C- | AVYYCARLSTGTFAYWGQGTLVTVSSASTKGPSVFPLAPCSRSTSE |
| terminal lysine | STAALGCLVKDYFPEPVTVSWNSGALTSGVHTFPAVLQSSGLYSLSS |
| | VVTVPSSSLGTKTYTCNVDHKPSNTKVDKRVESKYGPPCPPCPAPE |
| | FLGGPSVFLFPPKPKDTLMISRTPEVTCVVVDVSQEDPEVQFNWYV |
| | DGVEVHNAKTKPREEQFNSTYRVVSVLTVLHQDWLNGKEYKCKVSN |
| | KGLPSSIEKTISKAKGQPREPQVYTLPPSQEEMTKNQVSLTCLVKGF |
| | YPSDIAVEWESNGQPENNYKTTPPVLDSDGSFFLYSRLTVDKSRWQ |
| | EGNVFSCSVMHEALHNHYTQKSLSLSLG |
| | (SEQ ID NO: 2) |
| mAb7 full-length | DIVMTQSPDSLAVSLGERATINCKSSQSLWDSGNQKNFLTWYQQKP |
| light chain | GQPPKLLIYWTSYRESGVPDRFSGSGSGTDFTLTISSLQAEDVAVYY |
| | CQNDYFYPHTFGGGTKVEIKRGTVAAPSVFIFPPSDEQLKSGTASVV |
| | CLLNNFYPREAKVQWKVDNALQSGNSQESVTEQDSKDSTYSLSSTL |
| | TLSKADYEKHKVYACEVTHQGLSSPVTKSFNRGEC |
| | (SEQ ID NO: 3) |
| mAb7 light chain | QVQLVQSGAEVKKPGASVKVSCKASGYTFTSYWINWVRQAPGQGL |
| variable region | EWMGNIYPGSSLTNYNEKFKNRVTMTRDTSTSTVYMELSSLRSEDT |
| | AVYYCARLSTGTFAYWGQGTLVTVSS |
| | (SEQ ID NO: 4) |
| mAB7 and mAB15 | QVQLVQSGAEVKKPGASVKVSCKASGYTFTSYWINWVRQAPGQGL |
| heavy chain | EWMGNIWPGSSLTNYNEKFKNRVTMTRDTSTSTVYMELSSLRSEDT |
| variable region | AVYYCARLLTGTFAYWGQGTLVTVSS |
| | (SEQ ID NO: 5) |
| mAb15 light chain | DIVMTQSPDSLAVSLGERATINCKSSQSLWDSGNQKNFLTWYQQKP |
| variable region | GQPPKLLIYWTSYRESGVPDRFSGSGSGTDFTLTISSLQAEDVAVYY |
| | CQNDYFYPHTFGGGTKVEIK |
| | (SEQ ID NO: 6) |
| Nivolumab, | QVQLVESGGGWQPGRSLRLDCKASGITFSNSGMHWVRQAPGKGL |
| MDX1106, full | EWVAVrWYDGSKRYYADSVKGRFTISRDNSKNTLFLQMNSLRAEDT |
| length heavy chain | AVYYCATNDDYWGQGTLVTVSSASTKGPSVFPLAPCSRSTSESTAA |
| From | LGCLVDYFPEPVTVSWNSGALTSGVHTFPAVLQSSGLYSLSSVVTVP |
| WO2006/121168 | SSSLGTTYTCNVDHKPSNTKVDRVESYGPPCPPCPAPEFLGGPSVF |
| | LFPPKPKDTLMISRTPEVTCWVDVSQEDPEVQFNWYYDGVEVHNAT |
| | KPREEQFNSTYRVVSVLTVLHQDWLNGKEYKCKVSNKGLPSSIEKTI |
| | SKA |
| | GQPREPQVYTLPPSQEEMTKNQVSLTCLVKGFYPSDIAVEWESNGQ |
| | PEKNYKTTPPVLDSDGSFFLYSRLTVDKSRWQEGNVFSCSVMHEAL |
| | HNHYTQKSLSLSLGK |
| | (SEQ ID NO: 7) |
| Nivolumab, | EIVLTQSPATLSLSPGERATLSCRASQSVSSYLAWYQQPGQAPRLLI |
| MDX1106, full | YDASNRATGIPARFSGSGSGTDFTLTISSLEPEDFAVYYCQQSSNWP |
| length light chain | RTFGQGTKVEIRTVAAPSVFIFPPSDEQLSGTASVVCLLNNFYPREAV |
| From | QWKVDNALQSGNSQESVTEQDSDSTYSLSSTLTLSKADYEKHKVYA |
| WO2006/121168 | CEVTHQGLSSPVT SFNRGEC |
| | (SEQ ID NO: 8) |
| Pembrolizumab, | QVQLVQSGVEVKKPGASVKVSCKASGYTFTNYYMYWVRQAPGQ |
| MK3475, full length | GLEWMGGINPSNGGTNFNEKFKNRVTLTTDSSTTTAYMELKSLQ |
| heavy chain | FDDTAVYYCARRDYRFDMGFDYWGQGTTVTVSSASTKGPSVFP |
| From | LAPCSRSTSESTAALGCLVKDYFPEPVTVSWNSGALTSGVHTFP |
| WO2009114335 | AVLQSSGLYSLSSVVTVPSSSLGTKTYTCNVDHKPSNTKVDKRVE |
| | SKYGPPCPPCPAPEFLGGPSVFLFPPKPKDTLMISRTPEVTCVVV |
| | DVSQEDPEVQFNWYVDGVEVHNAKTKPREEQFNSTYRVVSVLT |
| | VLHQDWLNGKEYKCKVSNKGLPSSIEKTISKAKGQPREPQVYTLP |
| | PSQEEMTKNQVSLTCLVKGFYPSDIAVEWESNGQPENNYKTTPP |
| | VLDSDGSFFLYSRLTVDKSRWQEGNVFSCSVMHEALHNHYTQK |
| | SLSLSLGK |
| | (SEQ ID NO: 9) |
| Pembrolizumab, | EIVLTQSPATLSLSPGERATLSCRASKGVSTSGYSYLHWYQQKP |
| MK3475, full length | GQAPRLLIYLASYLESGVPARFSGSGSGTDFTLTISSLEPEDFAVY |
| light chain | YCQHSRDLPLTFGGGTKVEIKRTVAAPSVFIFPPSDEQLKSGTAS |
| From | VVCLLNNFYPREAKVQWKVDNALQSGNSQESVTEQDSKDSTYS |
| WO2009114335 | LSSTLTLSKADYEKHKVYACEVTHQGLSSPVT KSFNRGEC |
| | (SEQ ID NO: 10) |
| AMP224, without | LFTVTVPKELYIIEHGSNVTLECNFDTGSHVNLGAITASLQKVENDTSP |
| signal sequence | HRERATLLEEQLPLGKASFHIPQVQVRDEGQYQCIIIYGVA |
| From | WDYKYLTLKVKASYRKINTHILKVPETDEVELTCQATGYPLAEVSWP |
| WO2010027827 | NVSVPANTSHSRTPEGLYQVTSVLRLKPPPGRNFSCVFWNTHVREL |
| and | TLASIDLQSQMEPRTHPTWEPKSCDKTHTCPPCPAPELLGGPSVFLF |
| WO2011066342 | PPKPKDTLMISRTPEVTCWVDVSHEDPEVKFNWYVDGVEVHNAKTK |
| | PREEQYNSTYRWSVLTVLHQDWLNGKEYKCKVSNKALPAPIEKTISK |
| | AKGQPREPQVYTLPPSRDELTKNQV SLTCLVKGFY PSDIAVEWES |
| | NGQPENNYKT TPPVLDSDGS |
| | FFLYSKLTVDKSRWQQGNVFSCSVMHEALHNHYTQKSLSLSPGK |
| | (SEQ ID NO: 11) |
| YW243.55.S70 | EVQLVESGGGLVQPGGSLRLSCAASGFTFSDSWIHWVRQAPGKGL |
| heavy chain | EWVAWISPYGGSTYYADSVKGRFTISADTSKNTAYLQMNSLRAEDT |
| variable region | AVYYCARRHWPGGFDYWGQGTLVTVSA |
| From | (SEQ ID NO: 12) |
| WO2010077634 | |
| YW243.55.570 light | DIQMTQSPSSLSASVGDRVTITCRASQDVSTAVAWYQQKPGKAPKL |
| chain variable | LIYSASFLYSGVPSRFSGSGSGTDFTLTISSLQPEDFATYYCQQYLYH |
| region | PATFGQGTKVEIKR |
| From | (SEQ ID NO: 13) |
| WO2010077634 | |
| avelumab heavy | EVQLLESGGGLVQPGGSLRLSCAASGFTFSSYIMWVRQAPGKGLE |
| chain variable | WVSSIYPSGGITFYADKGRFTISRDNSKNTLYLQMNSLRAEDTAVYYC |
| region | ARIKLGTVTTVDYWGQ GTLVTVSS |
| From WO13079174 | (SEQ ID NO: 14) |
| avelumab light | QSALTQPASVSGSPGQSITISCIGTSSDVGGYNYVSWYQQHPGKAP |
| chain variable | KLMIYDVSNRPSGVSNRFSGSKSGNTASLTISGLQAEDEADYYCSSY |
| region | TSSSTRVFGTGTKVTVL |
| From WO13079174 | (SEQ ID NO: 15) |

The term “PD-1 binding antagonist” as used herein refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less non-dysfunctional. In some embodiments, the PD-1 binding antagonist is an anti-PD-1 antibody. In a specific aspect, a PD-1 binding antagonist is nivolumab. In another specific aspect, a PD-1 binding antagonist is pembrolizumab. In another specific aspect, a PD-1 binding antagonist is pidilizumab.

The term “PD-L1 binding antagonist” as used herein refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 binding antagonist is an anti-PD-L1 antibody. In a specific aspect, an anti-PD-L1 antibody is avelumab. In another specific aspect, an anti-PD-L1 antibody is atezolizumab. In another specific aspect, an anti-PD-L1 antibody is durvalumab. In another specific aspect, an anti-PD-L1 antibody is BMS-936559 (MDX-1105).

As used herein, an anti-human PD-L1 antibody refers to an antibody that specifically binds to mature human PD-L1. A mature human PD-L1 molecule consists

(SEQ ID NO: 16)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

Table 3 below provides the sequences of the anti-PD-L1 antibody avelumab for use in the treatment method, medicaments and uses of the present invention. Avelumab is disclosed as A09-246-2, in International Patent Publication No. WO2013/079174, the disclosure of which is hereby incorporated by reference in its entirety.

TABLE 3
ANTI-HUMAN PD-L1 MONOCLONAL ANTIBODY AVELUMAB
SEQUENCES

| | |
|---|---|
| Heavy chain | SYIMM (SEQ ID NO: 17) |
| CDR1 (CDRH1) | |
| Heavy chain | SIYPSGGITFY (SEQ ID NO: 18) |
| CDR2 (CDRH2) | |
| Heavy chain | IKLGTVTTVDY (SEQ ID NO: 19) |
| CDR3 (CDRH3) | |
| Light chain CDR1 | TGTSSDVGGYNYVS (SEQ ID NO: 20) |
| (CDRL1) | |
| Light chain CDR2 | DVSNRPS (SEQ ID NO: 21) |
| (CDRL2) | |
| Light chain CDR3 | SSYTSSSTRV (SEQ ID NO: 22) |
| (CDRL3) | |
| Heavy chain | EVQLLESGGGLVQPGGSLRLSCAASGFTFSSYIMMWVRQAP |
| variable region | GKGLEWVSSIYPSGGITFYADKGRFTISRDNSKNTLYLQMNS |
| (VR) | LRAEDTAVYYCARIKLGTVTTVDYWGQGTLVTVSS |
| | (SEQ ID NO: 14) |
| Light chain VR | QSALTQPASVSGSPGQSITISCTGTSSDVGGYNYVSWYQQH |
| | PGKAPKLMIYDVSNRPSGVSNRFSGSKSGNTASLTISGLQAE |
| | DEADYYCSSYTSSSTRVFGTGTKVTVL |
| | (SEQ ID NO: 15) |
| Heavy chain | EVQLLESGGGLVQPGGSLRLSCAASGFTFSSYIMMWVRQAP |
| | GKGLEWVSSIYPSGGITFYADTVKGRFTISRDNSKNTLYLQM |
| | NSLRAEDTAVYYCARIKLGTVTTVDYWGQGTLVTVSSASTKG |
| | PSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCDKTHTCPPCPAPELLGGPSVFLFPPKP |
| | KDTLMISRTPEVTCVVVDVSHEDPEVKFNWYVDGVEVHNAK |
| | TKPREEQYNSTYRVVSVLTVLHQDWLNGKEYKCKVSNKALP |
| | APIEKTISKAKGQPREPQVYTLPPSRDELTKNQVSLTCLVKGF |
| | YPSDIAVEWESNGQPENNYKTTPPVLDSDGSFFLYSKLTVDK |
| | SRWQQGNVFSCSVMHEALHNHYTQKSLSLSPGK |
| | (SEQ ID NO: 23) |
| Light chain | QSALTQPASVSGSPGQSITISCTGTSSDVGGYNYVSWYQQH |
| | PGKAPKLMIYDVSNRPSGVSNRFSGSKSGNTASLTISGLQAE |
| | DEADYYCSSYTSSSTRVFGTGTKVTVLGQPKANPTVTLFPPS |
| | SEELQANKATLVCLISDFYPGAVTVAWKADGSPVKAGVETTK |
| | PSKQSNNKYAASSYLSLTPEQWKSHRSYSCQVTHEGSTVEK |
| | TVAPTECS |
| | (SEQ ID NO: 24) |

The term “PD-L2 binding antagonists” as used herein refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less non-dysfunctional. In some embodiments, a PD-L2 binding antagonist is a PD-L2 immunoadhesin.

A “PARP inhibitor” or a “PARPi” is a molecule that inhibits the function of poly(adenosine diphosphate [ADP]-ribose) polymerase (PARP) to repair the single stranded breaks (SSBs) of the DNA. In some embodiments, a PARP inhibitor is a small molecule, which is an organic compound that has molecular weight less than 900 Daltons. In some embodiments, the PARP inhibitor is a polypeptide with molecular weight more than 900 Daltons. In some embodiments, the PARP inhibitor is an antibody. In some embodiments, the PARP inhibitor is selected from the group consisting of olaparib, niraparib, BGB-290, talazoparib, or any pharmaceutically acceptable salt of olaparib, niraparib, BGB-290 or talazoparib thereof. In an embodiment, the PARP inhibitor is talazoparib, or a pharmaceutically acceptable salt thereof and preferably a tosylate thereof. In an embodiment, the PARP inhibitor is talazoparib tosylate.

“DNA damage response defect positive”, or or “DDR defect positive”, as used herein, refer to a condition when an individual or the cancer tissue in the individual is identified as having either germline or somatic genetic alternations in at least one of the DDR genes, as determined by genetic analysis. As used herein, the DDR genes refer to any of those genes that were included in Table 3 of the supplemental material in Pearl et al., Nature Reviews Cancer 15, 166-180 (2015), the disclosure of which is hereby incorporated by reference in its entirety. Exemplary DDR genes include, without limitation, those as described in the below Table 4. Preferred DDR genes include, without limitation, BRCA1, BRCA2, ATM, ATR and FANC. Exemplary genetic analysis includes, without limitation, DNA sequencing, the FoundationOne genetic profiling assay (Frampton et al, Nature Biotechnology, Vol 31, No. 11, 1023-1030, 2013).

TABLE 4
Exemplary DDR genes

| Gene(s) | Description |
|---|---|
| MUTYH (MYH), | Base excision repair (BER) |
| PARP1 (ADPRT), PARP2 (ADPRTL2), | Poly(ADP-ribose) |
| PARP3 (ADPRTL3) | polymerase (PARP) |
| | enzymes that bind to DNA |
| MSH2, MSH6, MLH1, PMS2, | Mismatch excision repair |
| | (MMR) |
| RPA1, ERCC2 (XPD), ERCC4 (XPF) | Nucleotide excision repair |
| | (NER) |
| RAD51, RAD51B, RAD51D, XRCC2, | Homologous recombination |
| XRCC3, RAD52, RAD54L, BRCA1, | |
| RAD50, MRE11A, NBN (NBS1), | |
| FANCA, FANCC, BRCA2 (FANCD1), | Fanconi anemia |
| FANCD2, FANCE, FANCF, | |
| FANCG (XRCC9), FANCI (KIAA1794), | |
| FANCL, FANCM, PALB2 (FANCN), | |
| RAD51C (FANCO), | |
| NUDT1 (MTH1), | Modulation of nucleotide |
| | pools |
| POLD1, POLE, | DNA polymerases (catalytic |
| | subunits) |
| ATM | Genes defective in diseases |
| | associated with sensitivity to |
| | DNA damaging agents |
| ATR, CHEK1, CHEK2, TP53BP1 | Other conserved DNA |
| (53BP1) | damage response genes |

“Loss of heterozygosity score” or “LOH score” as used here in, refers to the percentage of genomic LOH in the tumor tissues of an individual. Percentage genomic LOH, and the calculation thereof are described in Swisher et al (The Lancet Oncology, 18(1):75-87, January 2017), the disclosure of which is incorporated herein by reference in its entirety. Exemplary genetic analysis includes, without limitation, DNA sequencing, Foundation Medicine's NGS-based T5 assay.

“Homologous recombination deficiency score” or “HRD score” as used here in, refers to the unweighted numeric sum of loss of heterozygosity (“LOH”), telomeric allelic imbalance (“TAI”) and large-scale state transitions (“LST”) in the tumor tissues of an individual. HRD score together with LOH and LOH score, and the calculation thereof are described in Timms et al, Breast Cancer Res 2014 Dec. 5; 16(6):475, Telli et al Clin Cancer Res; 22(15); 3764-73.2016, the disclosures of which are incorporated herein by reference in their entireties. Exemplary genetic analysis includes, without limitation, DNA sequencing, Myriad's HRD or HRD Plus assay (Mirza et al N Engl J Med 2016 Dec. 1; 375(22):2154-2164, 2016).

The term “tumor proportion score” or “TPS” as used herein refers to the percentage of viable tumor cells showing partial or complete membrane staining in an immunohistochemistry test of a sample. “Tumor proportion score of PD-L1 expression” used here in refers to the percentage of viable tumor cells showing partial or complete membrane staining in a PD-L1 expression immunohistochemistry test of a sample. Exemplary samples include, without limitation, a biological sample, a tissue sample, a formalin-fixed paraffin-embedded (FFPE) human tissue sample and a formalin-fixed paraffin-embedded (FFPE) human tumor tissue sample. Exemplary PD-L1 expression immunohistochemistry tests include, without limitation, the PD-L1 IHC 22C3 PharmDx (FDA approved, Daco), Ventana PD-L1 SP263 assay, and the tests described in international patent application PCT/EP2017/073712.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Another particular example of cancer includes renal cell carcinoma.

The term “treating”, as used herein, unless otherwise indicated, means reversing, alleviating, inhibiting the progress of, or preventing the disorder or condition to which such term applies, or one or more symptoms of such disorder or condition.

A “patient” to be treated according to this invention includes any warm-blooded animal, such as, but not limited to human, monkey or other lower-order primate, horse, dog, rabbit, guinea pig, or mouse. For example, the patient is human. Those skilled in the medical art are readily able to identify individual patients who are afflicted with non-small cell lung cancer and who are in need of treatment.

The terms “treatment regimen”, “dosing protocol” and dosing regimen are used interchangeably to refer to the dose and timing of administration of each therapeutic agent in a combination of the invention.

“Ameliorating” means a lessening or improvement of one or more symptoms as compared to not administering a treatment. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metatasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “objective response” refers to a measurable response, including complete response (CR) or partial response (PR). In some embodiments, the “objective response rate (ORR)” refers to the sum of complete response (CR) rate and partial response (PR) rate.

“Complete response” or “CR” as used herein means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease

In some embodiments, the anti-cancer effect of the method of treating cancer, including “objective response”, “complete response”, “partial response”, “progressive disease”, “stable disease”, “progression free survival”, “duration of response”, as used herein, are as defined and assessed by the investigators using RECIST v1.1 (Eisenhauer et al, Eur J of Cancer 2009; 45(2):228-47) in patients with locally advanced or metastatic solid tumors other than metastatic CRPC, and RECIST v1.1 and PCWG3 (Scher et al, J Clin Oncol 2016 Apr. 20; 34(12):1402-18) in patients with metastatic CRPC. The disclosures of Eisenhauer et al, Eur J of Cancer 2009; 45(2):228-47 and Scher et al, J Clin Oncol 2016 Apr. 20; 34(12):1402-18 are herein incorporated by references in their entireties. An exemplary documentation required for the determination of radiographic progression of CRPC is shown in Table 5 below.

TABLE 5
Criteria for Evidence of Radiographic Progression

| | | | Criteria to Document |
|---|---|---|---|
| Progression | Criteria for | Criteria to Confirm | Disease Progression on |
| detected | Progression | Progression | Confirmatory Scan |
| Week 8 | Bone lesions: 2 or more | Timing: At least | 2 or more new bone |
| | new lesions compared to | 6 weeks after | lesions on bone scan |
| | screening bone scan by | progression identified | compared to Week 8 |
| | PCWG3 | or at Week 16 visit | scan |
| | Soft tissue lesions: | No confirmatory scan | No confirmatory scan |
| | Progressive disease on | required for soft tissue | required for soft tissue |
| | CT or MRI by RECIST | disease progression | disease progression |
| | v1.1 | | |
| Week | Bone lesions: 2 or more | Timing: At least | Persistent or increase in |
| 16 or | new lesions on bone | 6 weeks after | number of bone lesions |
| after | scan compared to Week | progression identified | on bone scan compared |
| | 8 bone scan | or at next imaging | to prior scan |
| | | time point^{b} | |
| | Soft tissue lesions: | No confirmatory scan | No confirmatory scan |
| | Progressive disease on | required for soft tissue | required for soft tissue |
| | CT or MRI by RECIST | disease progression | disease progression |
| | v1.1 | | |

In some embodiments, the anti-cancer effect of the treatment, including “immune-related objective response” (irOR), “immune-related complete response” (irCR), “immune-related partial response” (irCR), “immune-related progressive disease” (irPD), “immune-related stable disease” (irSD), “immune-related progression free survival” (irPFS), “immune-related duration of response” (irDR), as used herein, are as defined and assessed by Immune-related response criteria (irRECIST, Nishino et. al. J Immunother Cancer 2014; 2:17) for patients with locally advanced or metastatic solid tumors other than patients with metastatic CRPC. The disclosure of Nishino et. al. J Immunother Cancer 2014; 2:17 is herein incorporated by reference in its entirety.

As used herein, “overall survival” (OS) refers to the percentage of individuals in a group who are likely to be alive after a particular duration of time.

By “extending survival” is meant increasing overall or progression-free survival in a treated patient relative to an untreated patient (i.e. relative to a patient not treated with the medicament).

As used herein, “drug related toxicity”, “infusion related reactions” and “immune related adverse events” (“irAE”), and the severity or grades thereof are as exemplified and defined in the National Cancer Institute's Common Terminology Criteria for Adverse Events v 4.0 (NCI CTCAE v 4.0).

As used herein, “in combination with” or “in conjunction with” refers to administration of one treatment modality in addition to another treatment modality. As such, “in combination with” or “in conjunction with” refers to administration of one treatment modality before, during, or after administration of the other treatment modality to the individual.

A “low-dose amount”, as used herein, refers to an amount or dose of a substance, agent, compound, or composition, that is lower than the amount or dose typically used in a clinical setting.

The term “advanced”, as used herein, as it relates to solid tumors, includes locally advanced (non-metastatic) disease and metastic disease. Locally advanced solid tumors, which may or may not be treated with curative intent, and metastatic disease, which cannot be treated with curative intent are included within the scope of “advanced solid tumors, as used in the present invention. Those skilled in the art will be able to recognize and diagnose advanced solid tumors in a patient.

“Duration of Response” for purposes of the present invention means the time from documentation of tumor model growth inhibition due to drug treatment to the time of acquisition of a restored growth rate similar to pretreatment growth rate.

The term “additive” is used to mean that the result of the combination of two compounds, components or targeted agents is no greater than the sum of each compound, component or targeted agent individually. The term “additive” means that there is no improvement in the disease condition or disorder being treated over the use of each compound, component or targeted agent individually.

The terms “synergy” or “synergistic” are used to mean that the result of the combination of two compounds, components or targeted agents is greater than the sum of each agent together. The terms “synergy” or “synergistic” means that there is an improvement in the disease condition or disorder being treated, over the use of each compound, component or targeted agent individually. This improvement in the disease condition or disorder being treated is a “synergistic effect”. A “synergistic amount” is an amount of the combination of the two compounds, components or targeted agents that results in a synergistic effect, as “synergistic” is defined herein. Determining a synergistic interaction between one or two components, the optimum range for the effect and absolute dose ranges of each component for the effect may be definitively measured by administration of the components over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as. benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma I I and calicheamicin omegaI I (see, e.g., Nicolaou et ai, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-it inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY 1 1 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRFI) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

A “chemotherapy” as used herein, refers to a chemotherapeutic agent, as defined above, or a combination of two, three or four chemotherapeutic agents, for the treatment of cancer. When a chemotherapy consists more than one chemotherapeutic agents, the chemotherapeutic agents can be administered to the patient on the same day or on different days in the same treatment cycle.

A “platinum-based chemotherapy” as used herein, refers to a chemotherapy wherein at least one chemotherapeutic agent is a coordination complex of platinum. Exemplary platinum-based chemotherapy includes, without limitation, cisplatin, carboplatin, oxaliplatin, nedaplatin, gemcitabine in combination with cisplatin, carboplatin in combination with pemetremed.

A “platinum-based doublet” as used herein, refers to a chemotherapy comprising two and no more than two chemotherapeutic agents and wherein at least one chemotherapeutic agent is a coordination complex of platinum. Exemplary platinum-based doublet includes, without limitation, gemcitabine in combination with cisplatin, carboplatin in combination with pemetrexed.

As used herein, the term “systemic anti-cancer therapy” refers to the systemic administration of pharmaceutical agent(s) approved by the regulatory agencies of any countries in the world, or in human clinical trials conducted under the regulatory agencies of any countries in the world, with the general intent to change the outcome of cancer. Systemic anti-cancer therapy includes, but is not limited to, chemotherapy, hormonal therapy, targeted anti-cancer therapy, cancer vaccines, oncolytic vaccines and adoptive T cell therapy.

As used herein, as describing the amount of carboplatin administered to the patient, the term “calculated AUC 3 dose”, “calculated AUC 4 dose”, “calculated AUC 5 dose”, “calculated AUC 6 dose”, etc, refers to the amount of carboplatin calculated according to the Calvert Equation based on the targeted area under the curve (AUC) being 3, 4, 5, and 6 mg-min/mL respectively and the patient's glomerular filtration rate (GFR, mL/min): carboplatin dose (mg)=target AUC (mg-min/mL)×(GFR+25), as described in the National Comprehensive Cancer Network® (NCCN) Chemotherapy Order Templates Appendix B as updated February 2018.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-Ia, IL-2, IL-3, IL-4, IL-5, IL-6, IL-7, IL-8, IL-9, IL10, IL-1 1, IL-12, IL-13, IL-15, IL-17A-F, IL-18 to IL-29 (such as IL-23), IL-31, including PROLEUKIN® rIL-2; a tumor-necrosis factor such as TNF-a or TNF-β, TGF-I-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

The phrase “pharmaceutically acceptable” indicates that the substance or composition must be compatible chemically and/or toxicologically, with the other ingredients comprising a formulation, and/or the mammal being treated therewith.

Some embodiments relate to the pharmaceutically acceptable salts of the compounds described herein. The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

The term “solvate” is used herein to describe a molecular complex comprising a compound described herein and one or more pharmaceutically acceptable solvent molecules, for example, water and ethanol.

The compounds described herein may also exist in unsolvated and solvated forms. Accordingly, some embodiments relate to the hydrates and solvates of the compounds described herein.

Compounds described herein containing one or more asymmetric carbon atoms can exist as two or more stereoisomers. Where a compound described herein contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds described herein containing, for example, an imino, keto, or oxime group, or so-called valence tautomerism in compounds which contain an aromatic moiety. A single compound may exhibit more than one type of isomerism.

The compounds of the embodiments described herein include all stereoisomers (e.g., cis and trans isomers) and all optical isomers of compounds described herein (e.g., R and S enantiomers), as well as racemic, diastereomeric and other mixtures of such isomers. While all stereoisomers are encompassed within the scope of our claims, one skilled in the art will recognize that particular stereoisomers may be preferred.

In some embodiments, the compounds described herein can exist in several tautomeric forms, including the enol and imine form, and the keto and enamine form and geometric isomers and mixtures thereof. All such tautomeric forms are included within the scope of the present embodiments. Tautomers exist as mixtures of a tautomeric set in solution. In solid form, usually one tautomer predominates. Even though one tautomer may be described, the present embodiments include all tautomers of the present compounds.

Included within the scope of the present embodiments are all stereoisomers, geometric isomers and tautomeric forms of the compounds described herein, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

The present embodiments also include atropisomers of the compounds described herein. Atropisomers refer to compounds that can be separated into rotationally restricted isomers.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where a compound described herein contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

Exemplary methods and materials are described herein, although methods and materials similar or equivalent to those described herein can also be used in the practice or testing of the invention. The materials, methods, and examples are illustrative only and not intended to be limiting.

### Methods, Uses, and Medicaments

In accordance with the present invention, an amount of a first compound or component, for example, a PARP inhibitor, is combined with an amount of a second compound or component, for example, a PD-1 axis binding antagonist, and the amounts together are effective in the treatment of non-small cell lung cancer. The amounts, which together are effective, will relieve to some extent one or more of the symptoms of the disorder being treated. In reference to the treatment of cancer, a effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression”.

In an embodiment, the invention is related to a method for treating cancer comprising administering to a patient in need thereof an amount of a PARP inhibitor in combination with an amount of a PD-1 axis binding antagonist, that is effective in treating cancer. In a further embodiment, the invention is related to a method for treating cancer comprising administering to a patient in need thereof an amount of a PARP inhibitor and an amount of a PD-1 axis binding antagonist, wherein the amounts together are effective in the cancer. In another embodiment, the invention is related to combination of a PARP inhibitor and a PD-1 axis binding antagonist, for use in the treatment of cancer. In another embodiment, the invention is related to a method for treating cancer comprising administering to a patient in need thereof an amount of a PARP inhibitor and an amount of a PD-1 axis binding antagonist, wherein the amounts together achieve synergistic effects in the treatment of cancer. In another embodiment, the invention is related to a combination of a PARP inhibitor and a PD-1 axis binding antagonist for the treatment of cancer, wherein the combination is synergistic. In an embodiment, the method or use of the invention is related to a synergistic combination of targeted therapeutic agents, specifically a PARP inhibitor and a PD-1 axis binding antagonist. In one aspect of all the embodiments of this paragraph, the PARP inhibitor is talazoparib or a pharmaceutically acceptable salt thereof and preferably a tosylate salt thereof, the PD-1 axis binding antagonist is avelumab

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the non-small cell lung cancer requiring treatment, and the presence of other medications.

In an embodiment, talazoparib, or a pharmaceutically acceptable salt thereof and preferably a tosylate thereof, is administered at a daily dosage of from about 0.1 mg to about 2 mg once a day, preferably from about 0.25 mg to about 1.5 mg once a day, and more preferably from about 0.5 to about 0.01 mg once a day. In an embodiment, talazoparib or a pharmaceutically acceptable salt thereof and preferably a tosylate thereof, is administered at a daily dosage of about 0.5 mg, 0.75 mg or 1.0 mg once daily. Dosage amounts provided herein refer to the dose of the free base form of talazoparib, or are calculated as the free base equivalent of an administered talazoparib salt form. For example, a dosage or amount of talazoparib, such as 0.5, 0.75 mg or 1.0 mg refers to the free base equivalent. This dosage regimen may be adjusted to provide the optimal therapeutic response. For example, the dose may be proportionally reduced or increased as indicated by the exigencies of the therapeutic situation.

The practice of the method of this invention may be accomplished through various administration or dosing regimens. The compounds of the combination of the present invention can be administered intermittently, concurrently or sequentially. In an embodiment, the compounds of the combination of the present invention can be administered in a concurrent dosing regimen.

Repetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

In an embodiment, the PARP inhibitor is talazoparib, or a pharmaceutically acceptable salt thereof and preferably a tosylate thereof, and is administered once daily to comprise a complete cycle of 28 days. Repetition of the 28 day cycles is continued during treatment with the combination of the present invention.

In an embodiment, talazoparib, or a pharmaceutically acceptable salt thereof and preferably a tosylate thereof, is administered once daily to comprise a complete cycle of 21 days. Repetition of the 21 day cycles is continued during treatment with the combination of the present invention.

In some embodiments, the PD-1 axis binding antagonist is avelumab and will be administered intravenously at a dose of about 1, 2, 3, 4, 5, 6, 7, 8, 9, 10, 11, 12, 13, 14, 15, 16, 17, 18, 19 or 20 mg/kg at intervals of about 14 days (±2 days) or about 21 days (±2 days) or about 30 days (±2 days) throughout the course of treatment. In some embodiment, avelumab is administered as a flat dose of about 80, 150, 160, 200, 240, 250, 300, 320, 350, 400, 450, 480, 500, 550, 560, 600, 640, 650, 700, 720, 750, 800, 850, 880, 900, 950, 960, 1000, 1040, 1050, 1100, 1120, 1150, 1200, 1250, 1280, 1300, 1350, 1360, 1400, 1440, 1500, 1520, 1550 or 1600 mg, preferably 800 mg, 1200 mg or 1600 mg at intervals of about 14 days (±2 days) or about 21 days (±2 days) or about 30 days (±2 days) throughout the course of treatment. In certain embodiments, a subject will be administered an intravenous (IV) infusion of a medicament comprising any of the PD-1 axis binding antagonists described herein. In certain embodiment, the subject will be administered a subcutaneous (SC) infusion of a medicament comprising any of the PD-1 axis binding antagonist described herein.

In some embodiments, the PD-1 axis binding antagonist is RN888 and will be administered subcutaneously at a dose of about 1, 2, 3, 4, 5, 6, 7 or 8 mg/kg at intervals of about 14 days (±2 days) or about 21 days (±2 days) or about 30 days (±2 days) throughout the course of treatment. In some embodiment, RN888 is administer as a flat dose of about 80, 150, 160, 200, 240, 250, 300, 320, 350, 400, preferably 300 mg at intervals of about 14 days (±2 days) or about 21 days (±2 days) or about 30 days (±2 days). In some embodiments, RN888 is administered subcutaneously in an amount of 300 mg Q4W.

Administration of the compounds of the combination of the present invention can be effected by any method that enables delivery of the compounds to the site of action. These methods include oral routes, intraduodenal routes, parenteral injection (including intravenous, subcutaneous, intramuscular, intravascular or infusion), topical, and rectal administration.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### EXAMPLE

### Example 1: Clinical Study of the Combination of Talazoparib and Avelumab

This Example illustrate a Phase 1 b/2, open label, multi-center clinical trial study of avelumab in combination with talazoparib in adult patients with locally advanced (primary or recurrent) or metastatic solid tumors including NSCLC, TNBC, HR+ breast cancer, recurrent platinum-sensitive ovarian cancer, urothelial cancer (UC), and castration-resistant prostate cancer (CRPC). Up to 296 patients in total will be enrolled into the study.

Phase 1 b Part of the Trial—Dose Level Cohorts

During the Phase 1 b portion of this study, patients with locally advanced or metastatic solid tumors, who meet eligibility criteria, will be treated with one of up to 3 different doses of talazoparib (0.5 mg, 0.75 mg or 1.0 mg) administered orally QD in combination with a fixed dose of avelumab 800 mg IV Q2W, and will be evaluated for dose limiting toxicities (DLTs). The avelumab and talazoparib combination will be administered in 28-day cycles. The DLT evaluation period will be 28 days (ie, Cycle 1). The target enrollment cohort size is 3-6 patients.

The starting dose level will be 1.0 mg talazoparib QD plus 800 mg avelumab Q2W. The dose levels of the combination to be evaluated are included in Table 6.

TABLE 6
Talazoparib and Avelumab Dose Levels for phase 1b

| | Talazoparib Dose | Avelumab Dose |
|---|---|---|
| Dose level | (Oral) | (IV) |
| D-0 | 1 mg QD | 800 mg Q2W |
| D-1 | 0.75 mg QD | 800 mg Q2W |
| D-2 | 0.5 mg QD | 800 mg Q2W |

D = day;
QD = once daily;
Q2W = every 2 weeks;
avelumab will be administered as a 1-hour IV infusion.

In Phase 1 b, patients without DLTs who withdraw from study treatment before receiving at least 75% of the planned dose of the investigational products in Cycle 1 for reasons other than treatment-related toxicity are not evaluable for DLT. Additional patients will be enrolled in the specific enrollment cohort to replace patients who are not considered DLT-evaluable.

The Phase 1 b portion is completed when at least 12 DLT-evaluable patients have been treated at the highest dose associated with DLT rate of less than 0.33. Early completion of the Phase 1 b portion can be reached when 9 or more DLT-evaluable patients have been treated at the same dose level with no occurrence of DLT, as the DLT rate of less than 0.33 will be met. Once the Phase 1b portion is completed and the recommended phase 2 dose of the combination is determined, the Phase 2 portion will be initiated. Approximately 12-36 patients are expected to be enrolled in Phase 1 b using the modified toxicity probability interval (mTPI) method.

Phase 2 Part of the Trial—Expansion Cohorts

The overall available data (including safety and preliminary anti-tumor activity) emerging from the Phase 1b portion of the study will be evaluated before starting enrollment of patients in the Phase 2 portion of the study. The Phase 2 portion of this study will further assess the safety and preliminary anti-tumor activity of the avelumab and talazoparib combination at the recommended phase 2 dose, which can be, but are not limited to the one of the three dosing regimens described in Table 6. Phase 2 expansion cohorts will include patients with locally advanced (primary or recurrent) or metastatic NSCLC, TNBC, HR+ breast cancer, ovarian cancer, UC, and CRPC, as described in more details in Table 7. Up to approximately 260 patients are expected to be enrolled in Phase 2.

TABLE 7
Expansion Cohorts in Phase 2 studies of Avelumab + Talazoparib

| | | Patient |
|---|---|---|
| Cohorts | Tumor type | Number |
| A1 | NSCLC | 40 |
| A2 | NSCLC, TPS no less than 50% for PD-L1 | 40 |
| B1 | TNBC | 20 |
| B2 | HR⁺ BC, DDR defect positive | 20 |
| C1 | Ovarian Cancer, recurrent platinum sensitive | 40 |
| C2 | Ovarian Cancer, recurrent platinum sensitive, | 20 |
| | BRCA gene defect | |
| D | Urothelial Cancer | 40 |
| E1 | CRPC | 20 |
| E2 | CRPC, DDR defect positive | 20 |

Key Patient Inclusion Criteria of the Study

Patients enrolled in the study must have histological diagnosis of locally advanced (primary or recurrent) or metastatic solid tumors that are not amenable for treatment with curative intent. Additional criterias are described in more details in the following.

For NSCLC in phase 1 b and phase 2 cohorts A1 and A2, the patients must have received 0-2 prior platinum-based chemotherapy regimens for locally advanced or metastatic NSCLC. If previously treated with platinum-based chemotherapy, the NSCLC patient must not have progressed while on treatment; disease progression after discontinuation of the platinum-based chemotherapy is allowed. The NSCLC patient must not have activating EGFR mutations, ALK translocations/rearrangements, or c-ros oncogene 1 (ROS1) translocations/rearrangements in the NSCLC. Non-squamous cell histologies require testing if status is unknown.

For NSCLC Cohort A2, patients must also have a previously documented tumor proportion score (TPS) 250% for PD-L1, determined through local laboratory testing using a 22C3 PD-L1 mAb or SP263 PD-L1 mAb based immunohistochemical assay on tumor tissue from a biopsy/surgery that was performed within 1 year prior to study enrollment, during which time the patient did not receive any intervening systemic anti-cancer treatment.

For the phase 1b TNBC cohort, patients must have received at least 1 prior chemotherapy regimen for locally advanced or metastatic breast cancer. There is no limit on the number of prior hormonal therapies or targeted anti-cancer therapies such as mammalian target of rapamycin (mTOR) or cyclin-dependent kinase (CDK) 4/6 inhibitors, or vascular endothelial growth factor (VEGF). If previously treated with neoadjuvant/adjuvant platinum-based chemotherapy, the phase 1 b TNBC patient must not have progressed while on treatment or within 6 months after stopping the platinum-based chemotherapy. If previously treated with a platinum-based chemotherapy in the advanced/metastatic setting, the phase 1 b TNBC patient must not have progressed while on treatment with the most recent platinum-based chemotherapy.

For the phase 2 TNBC cohort B1, patients must have received 0-2 prior chemotherapy regimens for locally advanced or metastatic breast cancer. There is no limit on the number of prior hormonal therapies or targeted anti-cancer therapies such as mammalian target of rapamycin (mTOR) or cyclin-dependent kinase (CDK)4/6 inhibitors, or vascular endothelial growth factor (VEGF). If previously treated with neoadjuvant/adjuvant platinum-based chemotherapy, the patient must not have progressed while on treatment or within 6 months after stopping the platinum-based chemotherapy. If previously treated with a platinum-based chemotherapy in the advanced/metastatic setting, the patient must not have progressed while on treatment with the most recent platinum-based chemotherapy.

For the phase 2 only hormone-Receptor-positive (HR+) Breast Cancer cohort B2, the patient must have DDR defect-positive disease, as determined by Foundation Medicine's Foundation One assay from FFPE tumor tissue submitted to the central laboratory. This tissue should be taken from the mandatory tumor biopsy acquired as part of this study or archival tumor tissue from a biopsy/surgery that was performed within 1 year prior to study enrollment, during which time the patient did not receive any intervening systemic anti-cancer treatment. The patient must have received 0-2 prior chemotherapy regimens for locally advanced or metastatic breast cancer following standard hormone therapy. There is no limit on prior hormonal therapies or targeted anti-cancer therapies such as mTOR or CDK4/6 inhibitors, or VEGF. If previously treated with neoadjuvant/adjuvant platinum-based, the patient must not have progressed while on treatment or within 6 months after stopping the platinum-based chemotherapy. If previously treated with a platinum-based chemotherapy in the advanced setting, the patient must not have progressed while on treatment with the most recent platinum-based chemotherapy.

For Recurrent Epithelial Ovarian Cancer patients in phase 1b, the patients must have been previously treated with at least 1 prior platinum-based chemotherapy regimen, with no disease progression while on treatment (platinum refractory), and disease progression within 6 months after stopping the last platinum-based chemotherapy (platinum resistant recurrent).

For the phase 2 Recurrent Epithelial Ovarian Cancer, cohorts C1 and C2, the patients must have been previously treated with 1-2 prior platinum-based chemotherapy regimens and received platinum-based chemotherapy as their last treatment; no disease progression while on treatment or within 6 months after stopping the last platinum-based chemotherapy, also termed “platinum sensitive recurrent disease”; For cohort C2, patients must have a germline or somatic BRCA1 or BRCA2 gene defect based on a previous test result from a clinical diagnostic test that is approved by the FDA (or an equivalent regulatory authority) at a local laboratory.

For the transitional Cell Carcinoma of the Urothelium (UC) including Bladder, Urethra, Ureters, or Renal Pelvis studies in the phase 1 b, patients must have received at least 1 prior systemic platinum-based chemotherapy regimen for locally advanced or metastatic UC or be ineligible for platinum-based chemotherapy. If previously treated with platinum based chemotherapy, the patient must not have progressed while on treatment; disease progression after discontinuation of the platinum-based chemotherapy is required.

For the transitional Cell Carcinoma of the Urothelium (UC) including Bladder, Urethra, Ureters, or Renal Pelvis studies phase 2, cohort D, the patient must have received 0-2 prior systemic platinum-based chemotherapy regimens for locally advanced or metastatic UC. If previously treated with platinum-based chemotherapy, the patient must not have progressed while on treatment; disease progression after discontinuation of the platinum-based chemotherapy is allowed.

For the CRPC without neuroendocrine differentiation, signet cell, or small cell features, in studies of Phase 1 b and Phase 2 cohorts E1 and E2, the patient must have metastatic disease. Patients with disease spread limited to regional pelvic lymph nodes (below the aortic bifurcation) are not eligible unless bone metastasis is present on bone scan. The patient must have received 1-2 prior chemotherapy regimens, including at least 1 taxane-based regimen for metastatic prostate cancer. The patient must have progressed on at least 1 line of novel hormonal therapy (enzalutamide and/or abiraterone acetate/prednisone) for treatment of metastatic CRPC. Serum testosterone≤1.73 nmol/L (50 ng/dL). Bilateral orchiectomy or ongoing androgen deprivation therapy with a gonadotropin-releasing hormone (GnRH) agonist/antagonist (surgical or medical castration) is required. The patient must have progressive disease at enrollment defined as 1 or more of the following 3 criteria: (1) A minimum of 3 rising PSA values with an interval of at least 1 week between determinations. The screening PSA value must be ≥2 μg/L (2 ng/mL) if qualifying solely by PSA progression; (2) Soft tissue disease progression as defined by RECIST v1.1, or (3) Bone disease progression defined by Prostate Cancer Working Group 3 (PCWG3) with 2 or more new metastatic lesions on bone scan.

For cohort E2, the patient's disease must also be DDR defect-positive, as determined by Foundation Medicines's Foundation One assay from FFPE tumor tissue submitted to the central laboratory. This tissue should be taken from the mandatory tumor biopsy acquired as part of this study or archival tumor tissue from a biopsy/surgery that was performed within 1 year prior to study enrollment, during which time the patient did not receive any intervening systemic anti-cancer treatment. For patients with no biopsable lesion outside of bone, archival tumor tissue from a biopsy/surgery performed within 5 years prior to study enrollment must be submitted. Tissue can be sent as slides or blocks.

Tumor Response Assessment

Anti-Tumor activity will be assessment through radiological tumor assessment of by CT or MRI at all known or suspected disease sites, such as chest, abdomen, pelvis, brain (if brain metastases confirmed or clinically suspected) or whole body. For all tumor types except for patient with CRPC, such assessment will be done at baseline, during treatment every 8 weeks for one year form the start of the study, and then every 16 weeks until disease progression regardless of initiation of subsequent anti-cancer therapy. In addition, bone scans (preferred method) or 18 fluorodeoxyglucose positron emission tomography (18F FDG PET)/CT or will be required at baseline, then every 16 weeks for the first year of study treatment and every 24 weeks thereafter, only if bone metastases are present at baseline.

For patient with CRPC, CT or MRI and bone scan at all known or suspected disease sites, such as chest, abdomen, pelvis, bone, brain (if brain metastases confirmed or clinically suspected) or whole body will be done at baseline, during treatment every 8 weeks for 24 weeks from the start of the study treatment, and then every 12 weeks thereafter until disease progression regardless of initiation of subsequent anti-cancer therapy.

Assessment of response for locally advanced or metasatic solid tumors except CRPC will be made using RECIST v1.1 and irRECIST. For CRPC, the investigators will assess response of soft tissue disease by RECIST v1.1. Bone disease will not be considered as non-target lesions assessed by RECIST v1.1, but will be assessed for progressive disease by PCWG3. The documentation required for the determination of radiographic progression of CRPC is shown in Table 5 titled “Criteria for Evidence of Radiographic Progression”.

For patients with ovarian cancer, blood will be collected on the first day of each treatment cycle of 28 days until the end of the treatment and analyzed at local laboratories for cancer antigen 125 (CA-125) testing to monitor the patient's disease. Elevated CA-125 test results should trigger a radiological tumor assessment due to suspected disease progression.

For patients with CRPC, blood will be collected on the first day of each treatment cycle of 28 days) until the end of the treatment and analyzed at local laboratories for prostate-specific antigen (PSA) to monitor the patient's disease. Elevated PSA test results should trigger a radiological tumor assessment due to suspected disease progression.

### Example 2

Phase 3 clinical study to evaluate efficacy and safety of avelumab in combination with chemotherapy followed by maintenance therapy of avelumab in combination with the PARP inhibitor talazoparib, and in particular, talazoparib tosylate, in previously untreated advanced ovarian cancer.

Approximately 720 patients with histologically confirmed stage III-IV epithelial ovarian, fallopian tube, or primary peritoneal cancer (according to American Joint Committee on Cancer (AJCC)/UICC TNM and International Federation of Gynecology and Obstetrics (FIGO) Staging System 2014 edition), untreated for the underlying cancer, including a minimum of 360 patients with tumors that with defects in DNA damage repair (DDR+), will be randomized to one of the arms as shown in Table 8:

TABLE 8
Study design

| | Chemotherapy treatment | Maintenance treatment |
|---|---|---|
| Arm A | Paclitaxel 175 mg/m² IV over 3 hrs | Avelumab 800 mg |
| | followed by Carboplatin AUC 5 or | administered IV on days 1, |
| | 6 IV over 15-60 minutes on day 1 | 15 and 29 of each 6-week |
| | of each 3 week cycle for 6 cycles | cycle |
| | Avelumab 800 mg IV on day 1 of | Talazoparib 1 mg orally |
| | each 3-week cycle for 6 cycles | once a day every day of |
| | | each 6-week cycle |
| Arm B | Paclitaxel 175 mg/m² IV over 3 hrs | Talazoparib 1 mg or 0.75 |
| | followed by Carboplatin AUC 5 or | mg orally once a day |
| | 6 IV over 15-60 minutes on day 1 | every day of each |
| | of each 3 week cycle for 6 cycles | 6-week cycle |
| Arm C | Paclitaxel 175 mg/m² IV over 3 hrs | Bevacizumab 15 mg/kg |
| | followed by Carboplatin AUC 5 or | administered IV on days |
| | 6 IV over 15-60 minutes on day 1 | 1 and 22 of each |
| | of each 3 week cycle for 6 cycles | 6-week cycle |
| | Bevacizumab 15 mg/kg IV on day | |
| | 1 of each 3-week cycle beginning | |
| | with cycle 2 for adjuvant patients, | |
| | and for neoadjuvant patients, on | |
| | day 1 of each 3-week cycle for | |
| | cycles 1, 2, 5 and 6 | |

Carboplatin dose = targeted AUC × (GFR + 25) per the Calvert Equation

Maintenance treatment in the above Table 8 should start within 4 weeks of the last dose of the chemotherapy in the chemotherapy treatment.

In the event of significant toxicity, dosing of each of the drug in the study may be interrupted, delayed or reduced as shown in Table 9.

TABLE 9
Dose level of Dose reduction

| | Starting dose | Reduced dose |
|---|---|---|
| Talazoparib | 1 mg daily | 0.75 mg daily, 0.5 mg daily |
| Talazoparib | 0.75 daily | 0.50 mg daily or 0.25 mg daily |
| Avelumab * | 800 mg Q3W | Temporarily or permanently |
| | | discontinue |
| Avelumab ** | 800 mg Q2W | Temporarily or permanently |
| | | discontinue |
| Paclitaxel | 175 mg/m² Q3W | 135 mg/m² Q3W |
| | | 110 mg/m² Q3W |
| Carboplatin | AUC 6 03W | AUC 5 Q3W or AUC 4 Q3W |
| Carboplatin | AUC 5 03W | AUC Q3W or AUC 3 Q3W |

* avelumab administered in the chemotherapy treatment
** avelumab administered in the maintenance treatment

Patient will continue to receive the maintenance treatment until progressive disease (PD) based on Blinded Independent Central Review (“BICR”) assessment per RECIST v1.1, unacceptable toxicity or withdrawal of consent. For the Arm A maintenance treatment, talazoparib and avelumab, the maximum duration of maintenance treatment is 24 months.

Assessment of response, including PFS, will be made using RECIST version 1.1 as assessed by BICR and investigator and as per irRECIST as assessed by investigator. Tumor tissue samples and blood samples will be obtained for each patient before the treatment, and at the end of the treatment. Blood samples of the patient will also be collected at various times during the treatment cycles. Retrospective DDR biomarker analysis will be conducted. Additional analysis such as PD-L1 expression, presence/absence of tumore infiltrating CD8+T lymphocytes, tumor mutational burden and lost of heterozygosity, presence of mutations in key oncogenes, presence of any proteomic or genetic signature, will be conducted.

1. A method for treating cancer comprising administering to a patient in need thereof an amount of a PARP inhibitor and an amount of a PD-1 axis binding antagonist, wherein the amounts together are effective in treating cancer.

2. The method of claim 1, wherein the PD-1 axis binding antagonist is a PD-L1 antibody.

3. The method of claim 2, wherein the PD-L1 antibody is avelumab.

4. The method of claim 1, wherein the PARP inhibitor is talazoparib, or a pharmaceutically acceptable salt thereof.

5. The method of claim 1, wherein the PARP inhibitor is talazoparib tosylate.

6. The method of claim 1, wherein the PD-1 axis binding antagonist is avelumab, the PARP inhibitor is talazoparib, or a pharmaceutically acceptable salt thereof, and the cancer is selected from the group consisting of non-small cell lung cancer, triple negative breast cancer, hormone receptor positive breast cancer, ovarian cancer, urothelial cancer and castration-resistant prostate cancer.

7. (canceled)

8. The method of claim 6, wherein the cancer is DNA damage response (DDR) defect positive in at least one DDR gene selected from BRCA1, BRCA2, ATM, ATR and FANC.

9-10. (canceled)

11. The method of claim 6, wherein the patient has a homologous recombination deficiency (HRD) score of about 20 or above, 25 or above, 30 or above, 35 or above, 40 or above, 42 or above, 45 or above, or 50 or above.

12. (canceled)

13. The method of claim 6, wherein the patient has a loss of heterozygosity (LOH) score of about 5% or more, 10% or more, 14% or more 15% or more, 20% or more, or 25% or more.

14. (canceled)

15. The method of claim 6, wherein the patient has a tumor proportion score of less than about 1%, or equal or over about 1%, 5%, 10%, 25%, 50%, 75% or 80% for PD-L1.

16. The method of claim 6, wherein the amount of avelumab is administered intravenously at about 10 mg/kg Q2W or about 800 mg Q2W and the amount of talazoparib, or a pharmaceutically acceptable salt thereof, is administered orally at a free base equivalent amount of about 0.5 mg, 0.75 mg or 1.0 mg QD.

17. A method for treating cancer, comprising administering to a patient in need thereof an amount of a PARP inhibitor, wherein the PARP inhibitor is talazoparib, or a pharmaceutically acceptable salt thereof, and an amount of avelumab, wherein the amount of avelumab is administered intravenously at about 10 mg/kg Q2W, 10 mg/kg Q1W, 10 mg/kg Q1W for 12 weeks followed by about 10 mg/kg Q2W, 800 mg Q2W, 1200 mg Q2W, or about 800 mg Q1W for 12 weeks followed by about 800 mg Q2W, and the amount of talazoparib, or a pharmaceutically acceptable salt thereof, is administered orally at a free base equivalent amount of about 0.5 mg, 0.75 mg or 1.0 mg QD.

18. (canceled)

19. The method of claim 17, wherein the cancer is non-small cell lung cancer.

20. (canceled)

21. The method of claim 17, wherein the cancer is ovarian cancer.

22-29. (canceled)

30. The method of claim 17, wherein the cancer is castration-resistant prostate cancer.

31-38. (canceled)

39. The method of claim 17, wherein the cancer is breast cancer.

40-48. (canceled)

49. The method of claim 1, further comprising administering to the patient an amount of a chemotherapeutic agent or radiotherapy, wherein the amounts together are effective in treating cancer.

50. The method of claim 1, wherein the PD-1 axis binding antagonist is RN888, the PARP inhibitor is talazoparib, or a pharmaceutically acceptable salt thereof, and the cancer is selected from the group consisting of non-small cell lung cancer, triple negative breast cancer, hormone receptor positive breast cancer, ovarian cancer, urothelial cancer and castration-resistant prostate cancer.

51-98. (canceled)

99. The method of claim 1, wherein the cancer is locally advanced or metastatic non-small cell lung cancer, the patient has received 0, 1 or 2 prior lines of platinum-based chemotherapy treatment for the locally advanced or metastatic NSCLC and had no progression while on such chemotherapy treatment, and that the cancer has no EFGR, ALK or ROS-1 genomic tumor aberrations.

100-110. (canceled)

111. A method for treating cancer comprising a first treatment regimen followed by a second treatment regimen, wherein
the first treatment regimen comprises administering to a patient in need thereof an amount of a chemotherapy and an amount of a PD-1 axis binding antagonist;
the second treatment regimen comprises administering to the patient in need thereof an amount of a PARP inhibitor and an amount of a PD-1 axis binding antagonist;
and the amounts together are effective in treating cancer.

112. The method of claim 111, wherein the first treatment regimen comprises administering to the patient in need thereof the amount of the chemotherapy and the amount of the PD-1 axis binding antagonist for at least one cycle of a first treatment cycle.

113-134. (canceled)


SEQUENCE LISTING (24 sequences)

SEQ ID NO: 1 - protein, 444 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 443 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 221 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 117 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 5 - protein, 117 aa, Artificial Sequence
  Synthetic Construct
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 6 - protein, 113 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 7 - protein, 432 aa, Homo sapiens
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 8 - protein, 208 aa, Homo sapiens
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 9 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 432 aa, Artificial Sequence
  Synthetic Construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 118 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 13 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 117 aa, Artificial Sequence
  Synthetic Construct
EVQLLESGGGLVQPGGSLRLSCAASGFTFSSYIMWVRQAPGKGLEWVSSIYPSGGITFYADKGRFTISRDNSKNTLYLQMNSLRAEDTAVYYCARIKLGTVTTVDYWGQGTLVTVSS

SEQ ID NO: 15 - protein, 110 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 16 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 17 - protein, 5 aa, Artificial Sequence
  Synthetic Construct
SYIMM

SEQ ID NO: 18 - protein, 11 aa, Artificial Sequence
  Synthetic Construct
SIYPSGGITFY

SEQ ID NO: 19 - protein, 11 aa, Artificial Sequence
  Synthetic Construct
IKLGTVTTVDY

SEQ ID NO: 20 - protein, 14 aa, Artificial Sequence
  Synthetic Construct
TGTSSDVGGYNYVS

SEQ ID NO: 21 - protein, 7 aa, Artificial Sequence
  Synthetic Construct
DVSNRPS

SEQ ID NO: 22 - protein, 10 aa, Artificial Sequence
  Synthetic Construct
SSYTSSSTRV

SEQ ID NO: 23 - protein, 450 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS